(19) United States
(12) Patent Application Publication
(10) Pub. No.: US 2010/0015713 A1
(43) Pub. Date: Jan. 21, 2010

(54) COMPONENTS OF CELL CULTURE MEDIA PRODUCED FROM PLANT CELLS

(76) Inventors: Scott Deeter, Fort Collins, CO (US); Joseph E. Schmidt, Davis, CA (US); Kenneth J. Mabery, Antelope, CA (US); Delia R. Bethell, West Sacramento, CA (US); Ning Huang, Davis, CA (US); Steve Clyde Pettit, Sacramento, CA (US)

Correspondence Address: ARENT FOX LLP, 1050 CONNECTICUT AVENUE, N.W., SUITE 400, WASHINGTON, DC, 20036, US

(21) Appl. No.: 11/922,984

(22) PCT Filed: Jun. 27, 2006
(86) PCT No.: PCT/US2006/025195

§ 371 Date: Jul. 23, 2009

Related U.S. Application Data
(60) Provisional application No. 60/694,236, filed on Jun. 28, 2005.

(51) Int. Cl. C12N 5/02

(52) U.S. Cl. 435/406; 435/404; 435/405

(57) ABSTRACT

The present invention comprises the production of components of cell culture media produced from plant cells and cell culture media containing the same. Heterologous DNA comprising genes encoding the desired component are introduced into plant cells, especially rice, which then produce the desired component. The component can be isolated from the plant cell and combined with other components to form the required cell culture medium.

This application claims priority benefit to U.S. provisional application Ser. No. 60/694,236 filed Jun. 28, 2005. The priority application is incorporated herein by reference in its entirety.

### 1. FIELD OF THE INVENTION

The present invention relates to the production and use of recombinant proteins produced by plant cells as components of cell culture media.

### 2. BACKGROUND OF THE INVENTION

Cell culture techniques allow animal or plant cells that are removed from tissues to grow when supplied with the appropriate nutrients and conditions. The cells are capable of dividing and can continue to grow until limited by some culture variables such as nutrient depletion or toxic buildup (Butler, M. & Jenkins, H., Nutritional aspects of growth of animal cells in culture, J of Biotechnol. (1989) 12: 97-110). Cell culture techniques have a number of applications including investigation of the normal physiology or biochemistry of cells (Balaban, B. & Urman, B., Embryo culture as a diagnostic tool, Reprod. Biomed. Online (2003) 7(6): 671-82), testing the effect of various chemical compounds or drugs on specific cell types (Farkas, D. & Tannenbaum, S. R., In vitro methods to study chemically-induced hepatotoxicity: a literature review, Curr. Drug Metab. (2005) 6(2): 111-25), studying the sequential or parallel combination of various cell types to generate artificial tissues (Wang et al., Cartilage tissue engineering with silk scaffolds and human articular chondrocytes, Biomaterials (2006)), and synthesizing valuable biologics from large scale cell cultures (Zeilinger et al., Three-dimensional co-culture of primary human liver cells in bioreactors for in vitro drug studies: effects of the initial cell quality on the long-term maintenance of hepatocyte-specific functions, Altern. Lab Anim. (2002) 30(5): 525-38). Cell culture techniques have also been used for in vitro fertilization (Blake et al., Protein supplementation of human IVF culture media, J Assist. Reprod. Genet. (2002) 19(3): 137-43; Bungum et al., Recombinant human albumin as protein source in culture media used for IVF: a prospective randomized study, Reprod. Biomed. Online (2002) 4(3): 233-6), stem cell research (Conley et al., Derivation, propagation and differentiation of human embryonic stem cells, Int. J Biochem. Cell Biol. (2004) 36(4): 555-67), vaccine production (Chuang et al., Pharmaceutical strategies utilizing recombinant human serum albumin, Pharm. Res. (2002) 19(5): 569-77; GlaxoSmithKline, Publisher. HAVRIX® (Hepatitis A Vaccine, Inactivated) —Prescribing Information (2005), available at http://us.gsk.com/products/assets/us_havrix.pdf; Innis et al., Protection against hepatitis A by an inactivated vaccine, JAMA (1994) 271(17): 1328-34; Merck, Publisher. PROQUAD®—Measles, Mumps, Rubella, and Varicella (Oka/Merck) Virus Vaccine Live—Prescribing Information (2005), available at http://www.merck.com/product/usa/pi_circulars/p/proquad/proquad_pi.pdf; Litwin, J., The growth of Vero cells in suspension as cell-aggregates in serum-free media, Cytotechnology (1992) 10(2): 169-74), tissue engineering including artificial skin (Atala, A., Future perspectives in reconstructive surgery using tissue engineering, Urol. Clin. North Am. (1999) 26(1): 157-65, ix-x; Sher, et al., Targeting perlecan in human keratinocytes reveals novel roles for perlecan in epidermal formation, J Biol. Chem. (2006) 281(8): 5178-87) and organs (Neronov et al., Integrity of endothelium in cryopreserved human cornea, Cryo Letters (2005) 26(2): 131-6; Han, et al., Interleukin-1alpha-induced proteolytic activation of metalloproteinase-9 by human skin, Surgery (2005) 138(5): 932-9) and gene and cell therapy (Chadd, H. E. & Chamow, S. M., Therapeutic antibody expression technology, Curr. Opin. Biotechnol. (2001) 12(2): 188-94).

The biologics encompass a broad range of cell products and include specific proteins or viruses that require animal cells for propagation. For example, therapeutic proteins such as monoclonal antibodies can be synthesized in large quantities by growing genetically engineered cells in large-scale cultures (Dewar et al., Industrial implementation of in vitro production of monoclonal antibodies, Ilar J (2005) 46(3): 307-13). The number of such commercially valuable biologics has increased rapidly over the last decade and has led to the present widespread interest in mammalian cell culture technology (Mizrahi, A., Biologicals produced from animal cells in culture—an overview, Biotechnol. Adv. (1988) 6(2): 207-20).

The major advantage of using cell culture for any of the above applications is the consistency and reproducibility of results that can be obtained from using a batch of clonal cells. The need for cell culture, especially at large scale, became apparent with the need for viral vaccines. Major epidemics of polio in the 1940s and 1950s promoted a lot of effort to develop an effective vaccine. In 1949, it was shown that poliovirus could be grown in cultures of human cells, which led to considerable interest in the development of large quantities of the polio vaccine using cell culture (Ligon, B. L., Thomas Huckle Weller MD: Nobel Laureate and research pioneer in poliomyelitis, varicella-zoster virus, cytomegalovirus, rubella, and other infectious diseases, Semin. Pediatr. Infect. Dis. (2002) 13(1): 55-63). The polio vaccine, produced from de-activated virus, became one of the first commercial products of cultured animal cells (Furesz, J., Developments in the production and quality control of poliovirus vaccines—Historical perspectives, Biologicals (2006)).

Many components of cell culture media are obtained from animal sources, including lactoferrin, transferrin and serum albumin. These components are extracted from animal sources, purified, and then combined with other inorganic ingredients to constitute cell culture media (Mizrahi, A. & Lazar, A., Media for cultivation of animal cells: an overview, Cytotechnology (1988) 1: 199-214). Cell culture components produced in this way have several undesirable features. For one, there is no way to ensure that extraneous animal products are completely excluded from the component, no matter how precise the purification techniques. Animal-derived components can carry the risk of contamination of pathogens of animal and human origin such as BSE, HIV and hepatitis virus (Chamberland et al., Emerging infectious disease issues in blood safety, Emerg. Infect. Dis. (2001) 7(3 Suppl): 552-3; Hepatitis C: A Brief Review, America's Blood Centers (October 1998) vol. 1, issue 3 (Strong, M. ed.), available at http://www.americasblood.org/index.cfm?fuseaction=display.showPage&pageID=133; Weinberg et al., Legal, financial, and public health consequences of HIV contamination of blood and blood products in the 1980s and 1990s, Ann. Intern. Med. (2002) 136(4): 312-9).

For another, small proteinaceous particles, such as prions, could be present and current scientific evidence suggests that even a very small number could result in disease. In a recent case, a healthy subject died from possible transmission of variant Creutzfeldt-Jakob disease from blood transfusion (Llewelyn et al., Possible transmission of variant Creutzfeldt-Jakob disease by blood transfusion, Lancet (2004) 363(9407): 417-21). This raises concerns regarding the consistent safety of blood and blood-derived products (Bird, S. M., Recipients of blood or blood products “at vCJD risk,” BMJ (2004) 328(7432): 118-9). Because of such safety concerns, regulators are encouraging replacing animal components with recombinant proteins in vaccine production and other applications of cell culture techniques (Egan, W. M., Bovine-derived products used in the manufacture and formulation of vaccines: current policies and issues for the future (2004), available at http://www.fda.gov/cber/summaries/pda092004we.pdf; European Medicine Agency (EMA), CPMP position statement on Crutzfeldt-Jakob disease and plasma-derived and urine-derived medicinal products (2003), available at http://www.emea.eu.int/pdfs/human/press/pos/287902en.pdf; World Health Organization (WHO), Variant Creutzfeldt-Jakob disease (2002), available at http://www.who.int/mediacentre/factsheets/fs180/en/). Further, many users may be hesitant to use products from cell culture media that contain animal components for religious or moral reasons (Sarkar, S., Use of animal products in vegetarians and others, Anaesthesia (2005) 60(5): 519-20).

Therefore, there is a need to produce components for incorporation into cell culture media that is not derived from, or produced by, animal sources, yet still retain their functional value for cell cultures.

### SUMMARY OF THE INVENTION

Accordingly, it is an objective of the invention to provide cell culture media or media supplements containing components derived from plant cells, which when used in cell culture, results in the same or improved growth and productivity of cells as compared to the use of media containing animal-derived components.

One embodiment of the invention comprises a cell culture media with at least one plant-produced heterologous protein as a cell culture media component.

Another embodiment of the invention comprises a method for producing an enhanced cell culture media, comprising producing at least one heterologous protein in a plant cell and combining the at least one plant-produced heterologous protein with a cell culture media.

Another embodiment of the invention comprises a cell culture media produced by a process comprising producing at least one heterologous protein in a plant cell and combining at least one plant-produced heterologous protein with a cell culture media.

Another embodiment of the invention discloses that in a cell culture media having one or more proteinaceous cell culture media components, the improvement comprising at least one plant-produced heterologous protein as a cell culture media component, wherein the heterologous protein is produced by the following process:

a) transforming a plant cell with a chimeric gene comprising:

(i) a promoter from the gene of a seed storage protein;

(ii) a first DNA sequence, operably linked to the promoter, encoding a seed storage protein, and

(iii) a second DNA sequence, operably linked to the promoter, encoding the heterologous protein, wherein the first and second DNA sequences are linked in translation frame and together encode a fusion protein comprising the storage protein and the heterologous protein; and

b) growing a plant from the transformed plant cell for a time sufficient to produce seeds containing the heterologous protein. The embodiment may optionally include purifying the heterologous protein from the harvested seeds.

Another embodiment of the invention comprises a method for producing a cell culture media having at least one plant-produced heterologous protein as a cell culture media component, the method comprising:

a) transforming a plant cell with a chimeric gene comprising:

(i) a promoter from the gene of a seed storage protein;

(ii) a first DNA sequence, operably linked to the promoter, encoding a seed storage protein, and

(iii) a second DNA sequence, operably linked to the promoter, encoding the heterologous protein, wherein the first and second DNA sequences are linked in translation frame and together encode a fusion protein comprising the storage protein and the heterologous protein;

b) growing a plant from the transformed plant cell for a time sufficient to produce seeds containing the heterologous protein;
c) harvesting the seeds from the plant; and
d) combining the heterologous protein with a cell culture media.

Another aspect of the invention comprises a method for creating a recombinant plant cell that can produce components that may be incorporated into cell culture media or supplement for cell culture media.

Another aspect of the invention comprises a method of transforming a plant cell by incorporating a DNA segment that encodes for a component of cell culture media.

Another aspect of the invention comprises a method of transforming a rice cell by incorporating a DNA segment that encodes for components of cell culture media such as amino acids, growth factors, and other cell culture proteins including lactoferrin and human serum albumin.

Another aspect of the invention comprises a cell culture medium with components that are derived from plants, and more preferably all the organic ingredients are derived exclusively from non-animal sources.

Another aspect of the invention comprises a method for the production of cell culture media, the method comprising incorporating into cell culture media components derived from recombinant plant cells.

Another aspect of the invention comprises of the use of the media supplemented with plant-derived components to improve cell growth and productivity in cell cultures.

Another aspect of the invention comprises improved cell culture media comprising one or more media ingredients and as a supplement to one or more cell culture media components derived from plant cells.

Another aspect of the invention comprises a method for achieving high growth rate of culture cells and high productivity of culture cells by culturing the culture cell in the improved cell culture media.

Another aspect of the invention comprises a method of producing an improved cell culture media comprising a) obtaining a plant cell transformed with a vector containing a plant-based promoter and a gene encoding a cell culture media component, b) cultivating the transformed plant cells to set seeds, c) harvesting the mature seeds, d) extracting and purifying of the cell culture components from the seed, and e) adding the components to cell culture media.

### BRIEF DESCRIPTION OF THE FIGURES

FIG. 1 is a comparison of the codon-optimized epidermal growth factor sequence (“Egfactor”) with a native epidermal growth factor sequence (“Native Gene”), aligned to show 53 codons in the mature sequences, with 27 (51%) codon changes and 30 (19%) nucleotide changes.

FIG. 2 is a restriction map of the 3,877 bp plasmid, API303 (pGt1-EGF v2.1), showing an expression cassette for epidermal growth factor (EGF), and containing a rice Gt1 promoter, a Gt1 signal peptide, codon optimized EGF, a Nos terminator and an ampicillin resistance selectable marker.

FIG. 3 is a restriction map of the 4,142 bp plasmid, API270 (pGlb-EGF v2.1), showing an expression cassette for epidermal growth factor (“EGF”), and containing a Glb promoter, a Glb signal peptide, codon optimized EGF, a Nos terminator and an ampicillin resistance selectable marker.

FIG. 4 is a Western blot analysis of recombinant human EGF (“rhEGF”) in the R1 generation of transgenic rice seeds. Lane 1 indicates extracts from seeds of control untransformed Taipei 309 rice variety (Oryza sativa, Japonica). Lanes 2 to 5 show rhEGF expressed in the seed extracts obtained from independent transgenic rice events. Lane 6 shows a purified rhEGF standard expressed in yeast, loaded at 125 ng. Lane 7 shows a broad range of molecular weight markers.

FIG. 5 is a comparison of the codon-optimized insulin-like growth factor I sequence (“Insgfact”) with a native human insulin-like growth factor I sequence (“native gene”), aligned to show 70 codons in the mature sequences, with 40 (57%) codon changes and 47 (22%) nucleotides changes.

FIG. 6 is a restriction map of the 4,194 bp plasmid, API271 (pGlb-IGF v2.1), showing an expression cassette for insulin-like growth factor I (“IGF”), and containing a Glb promoter, a Glb signal peptide, codon optimized IGF, a Nos terminator and an ampicillin resistance selectable marker.

FIG. 7 is a restriction map of the 3,928 bp plasmid, API304 (pGt1-IFG v2.1), showing an expression cassette for insulin-like growth factor I (“IGF”), and containing a rice Gt1 promoter, a Gt1 signal peptide, codon optimized IGF, a Nos terminator and an ampicillin resistance selectable marker.

FIG. 8 is a Western blot analysis of recombinant human IGF-I (“rhIGF”) expressed in the R1 generation of transgenic rice seeds. Lane 1 shows rice seed extract from seeds of control untransformed rice variety Taipei 309. Lanes 2 to 8 show rhIGF expressed in seed extracts obtained from seven independent transgenic rice events. Lane 9 shows a purified rhIGF-1 standard expressed in yeast, loaded at 1 μg. Lane 10 shows a broad range of molecular weight markers.

FIG. 9 is a comparison of the codon-optimized intestinal trefoil factor sequence (“Trefoil”) with a native intestinal trefoil factor sequence (“Native Gene”), aligned to show 60 codons in the mature sequences, with 26 (43%) codon changes and 28 (15%) nucleotide changes.

FIG. 10 is a restriction map of the 4,163 bp plasmid, API269 (pGlb-ITF-nos), showing an expression cassette for intestinal trefoil factor (“ITF”), and containing a Glb promoter, a Glb signal peptide, codon optimized ITF, a Nos terminator and an ampicillin resistance selectable marker.

FIG. 11 is a restriction map of the 3,889 bp plasmid, API307 (pGt1-ITF-nos), showing an expression cassette for intestinal trefoil factor (ITF), and containing a rice Gt1 promoter, a Gt1 signal peptide, codon optimized ITF, a Nos terminator and an ampicillin resistance selectable marker.

FIG. 12 is a Western blot analysis of recombinant human ITF (“rhITF”) expression in the R1 generation of transgenic rice seeds. Lane 1 indicates extracts from seeds of control untransformed Taipei 309 rice variety. Lanes 2 and 3 show rhITF expressed in the seed extracts obtained from two independent transgenic rice events. Lane 4 indicates a purified rhITF standard expressed in yeast, loaded at 1 μg. Lane 5 shows a broad range of molecular weight markers.

FIG. 13 is a comparison of the codon-optimized human lactoferrin sequence (“cod opt lac”) with a native human lactoferrin sequence (“native lac”), aligned to show 693 codons, with 413 (59.6%) codon changes and 467 (22.5%) nucleotide changes.

FIG. 14 is a plasmid map of the 5,817 bp plasmid, API164 (GT1-Lac), showing an expression cassette for human lactoferrin and containing a Gt1 promoter, a Gt1 signal peptide, codon optimized human lactoferrin, a Nos terminator and a kanamycin resistance selectable marker.

FIG. 15 shows the results of a SDS-PAGE analysis for the expression of recombinant human lactoferrin. Total proteins from rice seed extracts were suspended in Laemli sample buffer, run on a gradient gel and stained with Coomassie blue. Lane 1 is the molecular weight marker. Lanes 3 to 6 are purified human derived lactoferrin (Sigma Chemical, USA). Lanes 8 to 13 are single seed extracts from homozygous independent transgenic rice lines and lane 14 is a seed extract from non-transformed rice variety Taipei 309.

FIG. 16 is a stable expression of recombinant human lactoferrin in transgenic rice grains from R₂ through R₁₀ generations. Total soluble proteins from 1 g of brown rice flour was extracted with 40 ml of extraction buffer and clarified by centrifugation. The extract was analyzed via ELISA. Extraction was repeated three times and standard deviation is shown as an error bar.

FIG. 17 shows DNA and protein sequence of fusion between Gt1 signal peptide (Gt1 SP) and codon-optimized human serum albumin (OPTIMIZED HSA) sequence based on native protein sequence derived from P02768 (Swiss-Prot).

FIG. 18 is a plasmid map of the 5,496 bp plasmid, API504 (GT1-HSA), showing an expression cassette for human serum albumin and containing a Gt1 promoter, a Gt1 signal peptide, codon optimized human serum albumin, a Nos terminator and a kanamycin resistance selectable marker.

FIG. 19 is a plasmid map of the 12,388 bp plasmid, API508 (JH/GT1-HSA), showing an expression cassette for human serum albumin and containing a Gt1 promoter, a Gt1 signal peptide, codon optimized human serum albumin, a Nos terminator and a kanamycin resistance selectable marker.

FIG. 20 shows the effect of three forms of recombinant human lactoferrin on cell growth. The three forms of lactoferrin are asis-lactoferrin, apo-lactoferrin and holo-lactoferrin. Asis-lactoferrin is the same as partial-lactoferrin with approximately 50% iron saturation. The baseline is with basal media and the rest is baseline supplemented with various concentration of recombinant human lactoferrin as indicated in the graph. Cell growth is measured as thymidine incorporation.

FIG. 21 shows the effect of three forms of recombinant human lactoferrin on cell growth. The three forms of lactoferrin are asis-lactoferrin, apo-lactoferrin and holo-lactoferrin. Asis-lactoferrin is the same as partial-lactoferrin with approximate 50% iron saturation. The baseline is with basal media plus 5% fetal calf serum and the rest are baseline supplemented with epidermal growth factor, 5% FCS or recombinant human lactoferrin at a final concentration of 1 mg/ml. Cell growth is measured as number of cells/ml.

FIG. 22 shows cell culture media supplemented with recombinant human lactoferrin (LACROMIN™) expressed in rice grain promotes hybridoma cell growth and increases hybridoma cell number as compared to animal derived transferrin. FBS is fetal bovine serum. AFM6 is serum-free cell culture media derived from KC Bio, Kansas. AFM6-Fe is a serum-free media without iron.

FIG. 23 shows the effect of recombinant human serum albumin (rHSA) and plasma-derived human serum albumin (pHSA) on the growth of hybridoma AE1 cells in control medium (CM) containing reduced serum (1% FBS).

FIG. 24 shows the effect of recombinant human serum albumin (rHSA) and plasma-derived human serum albumin (pHSA) on the growth of hybridoma AE1 cells in serum-free medium (SMF).

### DETAILED DESCRIPTION

Unless otherwise indicated, all terms used herein have the meanings given below or are generally consistent with same meaning that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y., Ausubel F M et al. (1993) Current Protocols in Molecular Biology, John Wiley & Sons, New York, N.Y., and Gelvin and Schilperoot, eds. (1997) Plant Molecular Biology Manual, Kluwer Academic Publishers, The Netherlands for definitions and terms of the art.

General and specific techniques for producing proteins from plant cells may be obtained from the following patents and applications, each of which is incorporated herein in its entirety by reference: U.S. Pat. Appl. Pub. No. 2003/0172403 A1 (“Plant Transcription Factors and Enhanced Gene Expression”); U.S. Pat. No. 6,991,824 (“Expression of Human Milk Proteins in Transgenic Plants”); U.S. Pat. Appl. Pub. No. 2003/0221223 (“Human Blood Proteins Expressed in Monocot Seeds”); U.S. Pat. Appl. Pub. No. 2004-0078851 (“Production of Human Growth Factors in Monocot Seeds”); U.S. Pat. Appl. Pub. No. 2004/0063617 (“Method of Making an Anti-infective Composition for Treating Oral Infections”); and international application no. PCT/US2004/041083 (“High-level Expression of Fusion Polypeptides in Plant Seeds Utilizing Seed-Storage Proteins as Fusion Carriers”). Other general and specific techniques for producing proteins from plant cells may be obtained, for example, from the following references, each of which is incorporated herein in its entirety by reference: U.S. Pat. No. 5,693,507, U.S. Pat. No. 5,932,479, U.S. Pat. Nos. 6,642,053, and 6,680,426 (each titled “Genetic Engineering of Plant Chloroplasts”); U.S. Pat. Appl. Pub. No. 2005/0066384 (“Site-Targeted Transformation Using Amplification Vectors”); U.S. Pat. Appl. Pub. No. 2005/0221323 (“Amplification Vectors Based on Trans-Splicing”); U.S. Pat. Appl. Pub. No. 2006/0026718 (“Method of Controlling Cellular Processes in Plants”); and U.S. Pat. Appl. Pub. No. 2006/0075524 (Method of Controlling A Cellular Process in a Multi-Cellular Organism”); Marillonnet et al., Systemic Agrobacterium tumefaciens-mediated transfection of viral replicons for efficient transient expression in plants, Nature Biotech. (2005) 23(6): 718-723.

The polynucleotides of the invention may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

The term “stably transformed” with reference to a plant cell means the plant cell has a non-native (heterologous) nucleic acid sequence integrated into its genome which is maintained through two or more generations.

By “host cell” is meant a cell containing a vector and supporting the replication and/or transcription and/or expression of the heterologous nucleic acid sequence. Preferably, according to the invention, the host cell is a plant cell. Other host cells may be used as secondary hosts, including bacterial, yeast, insect, amphibian or mammalian cells, to move DNA to a desired plant host cell.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

The term “mature plant” refers to a fully differentiated plant.

The term “seed product” includes, but is not limited to, seed fractions such as de-hulled whole seed, flour (seed that has been de-hulled by milling and ground into a powder) a seed extract, preferably a protein extract (where the protein fraction of the flour has been separated from the carbohydrate fraction), malt (including malt extract or malt syrup) and/or a purified protein fraction derived from the transgenic grain.

The term “biological activity” refers to any biological activity typically attributed to that protein by those skilled in the art.

“Monocot seed components” refers to carbohydrate, protein, and lipid components extractable from monocot seeds, typically mature monocot seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Maturation-specific protein promoter” refers to a promoter exhibiting substantially up-regulated activity (greater than 25%) during seed maturation.

The term “growth factor” refers to proteins, or biologically active fragments thereof, including, without limitation, epidermal growth factor (EGF), keratinocyte growth factors (KGF) including KGF-1 and KGF-2, insulin-like growth factors (IGF) including IGF-I and IGF-II, intestinal trefoil factor (ITF), transforming growth factors (TGF) including TGF-α and -β1-3, granulocyte colony-stimulating factor (GCSF), nerve growth factor (NGF) including NGF-β, and fibroblast growth factor (FGF) including FGF-1-19 and -12β, and biologically active fragments of these proteins. The sequences of these and other growth factors are well-known to those of ordinary skill in the art.

“Heterologous DNA” refers to DNA which has been introduced into plant cells from another source, or which is from a plant source, including the same plant source, but which is under the control of a promoter that does not normally regulate expression of the heterologous DNA.

“Heterologous protein” is a protein encoded by a heterologous DNA. The proteins include, but are not limit to, growth factor(s), lactoferrin, transferrin, albumin, insulin, and fractions thereof, growth hormone and fractions thereof, Fibronectin (human)-attachment factor, Lamin (Mouse)-attachment factor, collagenase, platelet derived growth factor, Human brain-derived neurotrophic factor (BDNF), glial-derived neurotrophic factor (GDNF), thymic factors, haptocorin, lactahedrin, lactoperoxidase. Alpha-fetoprotein, immunoglobin, alpha-lactalbumin.

As used herein, the terms “native” or “wild-type” relative to a given cell, protein, polypeptide, nucleic acid, trait or phenotype, refers to the form in which that is typically found in nature.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, and chromatography. The results of any such purifying or isolating step(s) may still contain other components as long as the results enrich for the component of interest.

As used herein, the terms “transformed” or “transgenic” with reference to a host cell means the host cell contains a non-native or heterologous or introduced nucleic acid sequence that is absent from the native host cell. Further, “stably transformed” in the context of the present invention means that the introduced nucleic acid sequence is maintained through two or more generations of the host, which is preferably (but not necessarily) due to integration of the introduced sequence into the host genome.

“Cell culture media” refers to media for cell culture purpose which includes complete media, basal media or basal media supplemented with cell culture media component, cell culture media supplement, or media with various amounts of serum or chemically defined media.

“Cell culture media component” refers any heterologous proteins for use as a supplement to cell culture media.

“Cell culture media ingredient” includes cell culture media components, proteins, peptides, hormones, carbohydrates, amino acids, lipids, vitamins, antibiotics, organic and inorganic salts.

“Cell culture media supplement” refers to a combination of one or multiple cell culture media components with or without other ingredients for addition to cell culture media.

Most cells in culture grow best at 37° C. and at pH 7.4 with appropriate media. Typically, cell culture media contains carbohydrates, amino acids, various salts, bicarbonate to maintain pH, vitamins and hormones, and phenol red as pH indicator. Examples of cell culture media that can be formed include: Dulbecco's Modified Eagle's Medium (DME), Ham's Nutrient Mixtures, MCDB Media, Minimum Essential Medium Eagle, RPMI Media, Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, and William's Medium E.

Frequently, cell culture media is supplemented with serum at concentration of 5 to 10%. Serum is a complex mixture containing undefined essential matter for cell growth. Commonly used serum is fetal calf serum (FCS) and fetal bovine serum (FBS). Due to its nature of complex mixture, lack of consistency and potential risk of pathogen contamination, cell culture industries and regulatory agencies are seeking alternatives to adding serum to cell culture media.

Since cells grow poorly in basal media, the basal media is then supplemented with various growth factors, hormones, and other essential components to generate serum-free media but maintaining the same cell growth rate as in serum-based media. Human transferrin or bovine transferrin is added as a source of iron. Typically, serum-free media contains insulin, serum albumin, transferrin, insulin-like growth factor and epidermal growth factor. Other proteins such as lactoferrin have also been shown to be able to promote cell growth. Each component provides a distinct function in cell growth. In the art, these protein components are animal-derived. With the increase in the concern of potential pathogen contamination from animal sourced material, cell culture industries are trying to develop animal-component-free media. While some successes have been made with the use of plant-based hydrolysates or recombinant protein produced with microbial system, plant-based hydrolysate is an undefined mixture of components and recombinant protein from microbial sources are too expensive to use in routine cell culture. An alternative source of non-animal-derived components is urgently needed for cell culture industries.

The cell culture media components produced by the methods to be described in the following sections are entirely plant-based and may be combined with inorganic salts such as NaCl, KCl, NaH₂PO₄, NaHCO₃, CaCl₂, and MgCl₂ and other ingredients such as amino acids, vitamins, growth factors, sugars, and antibiotics to form a variety of different cell culture media.

Expression vectors for use in the present invention are chimeric nucleic acid constructs (or expression vectors or cassettes), designed for operation in plants, with associated upstream and downstream sequences.

In general, expression vectors for use in practicing the invention include the following operably linked elements that constitute a chimeric gene: a promoter derived from a gene encoding a plant protein, operatively linked to a gene encoding a heterologous protein. The vector can include a promoter from the gene of a maturation-specific monocot plant storage protein, a first DNA sequence, operably linked to the promoter, encoding a monocot plant seed-specific signal sequence (such as an N-terminal leader sequence or a C-terminal trailer sequence) capable of targeting a polypeptide linked thereto to an endosperm cell, preferably an endosperm-cell organelle, such as a protein storage body, and a second DNA sequence, linked in translation frame with the first DNA sequence, encoding a cell culture media component. The signal sequence is preferably cleaved from the cell culture media component in the plant cell.

The chimeric gene, in turn, is typically placed in a suitable plant-transformation vector having (i) companion sequences upstream and/or downstream of the chimeric gene which are of plasmid or viral origin and provide necessary characteristics to the vector to permit the vector to move DNA from bacteria to the desired plant host; (ii) a selectable marker sequence; and (iii) a transcriptional termination region generally at the opposite end of the vector from the transcription initiation regulatory region.

Numerous types of appropriate expression vectors, and suitable regulatory sequences are known in the art for a variety of plant host cells. The promoter region is chosen to be regulated in a manner allowing for induction under seed-maturation conditions. In one aspect of this embodiment of the invention, the expression construct includes a promoter which exhibits specifically up-regulated activity during seed maturation. Promoters for use in the invention are typically derived from cereals such as rice, barley, wheat, oat, rye, corn, millet, triticale or sorghum. Examples of such promoters include the maturation-specific promoter region associated with one of the following maturation-specific monocot plant storage proteins: rice glutelins, oryzins, and prolamines, barley hordeins, wheat gliadins and glutelins, maize zeins and glutelins, oat glutelins, and sorghum kafirins, millet pennisetins, and rye secalins.

One embodiment of the present invention involves high-level expression of heterologous molecules that are components of cell culture media. These components can be expressed in a plant cell by a chimeric gene comprising the gene(s) encoding the desired component operably linked with an endogenous plant protein encoding gene. In a preferred embodiment the endogenous plant encoding gene is a rice seed storage protein gene for expression in a rice mature seed expression system.

The expressed polypeptide may be a multi-domain polypeptide with one domain being the heterologous or exogenous cell culture component and the other being the endogenous plant protein. At least one selective purification tag and/or at least one specific protease cleavage site may be provided for eventual release of the cell culture media component from the monocot seed storage protein carrier. For example, a strategic methionine or tryptophan residue providing a chemical cleavage site may be engineered in frame between the domains for release of the cell culture component from the endogenous plant protein. This technique is useful for cell culture media components that have protein portions or are proteins, for example lactoferrin, human serum albumin, and human growth factors.

Other selective protease cleavage sites include, but are not limited to enterokinase (ek), Factor Xa, thrombin, V8 protease, GENENASE™ (a variant of subtilisin BPN′), α-lytic protease or tobacco etch virus protease. Alternatively, cleavage of the fusion protein could be performed via chemical cleaving agents such as cyanogen bromide or N-chlorosuccinimide.

The invention provides cell culture media components recombinantly produced in host monocot plant seed wherein the cell culture media component expressed comprises about 3% or greater of the total soluble protein in the monocot seed. Thus, for example, the yield of total soluble protein which comprises the cell culture media component targeted for production can be from about 3% to about 5%, from about 5% to about 10%, from about 10% to about 15%, from about 15% to about 20%, from about 20% to about 25%, and from about 25% to about 30% of the total soluble protein found in the recombinantly engineered production plant seed. Additionally, for example, the yield of total soluble protein which comprises the cell culture media component targeted for production can be about 3% or greater, about 5% or greater, about 10% or greater, about 15% or greater, about 20% or greater, and about 25% or greater.

An embodiment of the present invention is a cell culture media, comprising at least one plant-produced heterologous protein as a cell culture media component. In some embodiments, the plant is a monocot plant, such as rice, barley, wheat, rye, corn, millet, triticale, or sorghum, preferably rice. The heterologous protein is a plant protein or a non-plant protein, preferably human, which may be selected in some embodiments from the group consisting of growth factors, lactoferrin, transferrin, serum albumin, insulin, growth hormone, and fractions thereof, fibronectin attachment factor, lamin attachment factor, collagenase, platelet derived growth factor, brain-derived neutrophic factor, glial-derived neurotrophic factor, thymic factors, haptocorin, lactahedrin, lactoperoxidase, alpha-fetoprotein, immunoglobin, or alpha-lactalbumin. In some embodiments of the present invention, the growth factors are preferably epidermal growth factors, keratinocyte growth factors, insulin-like growth factors, intestinal trefoil factors, transforming growth factors, granulocyte colony-stimulating factors, nerve growth factors, fibroblast growth factors, or biologically active fragments thereof.

In some embodiments, the cell culture media is a reduced serum or serum-free medium. In other embodiments, the cell culture media is disclosed as a complete media, basal media, or basal media supplemented with a cell culture media component.

Another embodiment of the present invention is a method for producing a cell culture media, comprising producing at least one heterologous protein in a plant cell and combining the at least one plant-produced heterologous protein with a cell culture media.

Another embodiment of the present invention is a cell culture media produced by a process comprising producing at least one heterologous protein in a plant cell and combining at least one plant-produced heterologous protein with a cell culture media.

Another embodiment of the present invention is an improved cell culture media having one or more proteinaceous cell culture media components, the improvement comprising at least one plant-produced heterologous protein as a cell culture media component, wherein the heterologous protein is produced by the following process:

a) transforming a plant cell with a chimeric gene comprising:

(i) a promoter from the gene of a seed storage protein;

(ii) a first DNA sequence, operably linked to the promoter, encoding a seed storage protein, and

(iii) a second DNA sequence, operably linked to the promoter, encoding the heterologous protein, wherein the first and second DNA sequences are linked in translation frame and together encode a fusion protein comprising the storage protein and the heterologous protein; and

b) growing a plant from the transformed plant cell for a time sufficient to produce seeds containing the heterologous protein. In some embodiments, the heterologous protein is purified from the harvested seeds. In some embodiments, the heterologous protein constitutes at least about 3.0% of the total soluble protein in the harvested seeds.

Another embodiment of the present invention is a method for producing a cell culture media having at least one plant-produced heterologous protein as a cell culture media component, the method comprising:

a) transforming a plant cell with a chimeric gene comprising:

(i) a promoter from the gene of a seed storage protein;

(ii) a first DNA sequence, operably linked to the promoter, encoding a seed storage protein, and

(iii) a second DNA sequence, operably linked to the promoter, encoding the heterologous protein, wherein the first and second DNA sequences are linked in translation frame and together encode a fusion protein comprising the storage protein and the heterologous protein;

b) growing a plant from the transformed plant cell for a time sufficient to produce seeds containing the heterologous protein;
c) harvesting the seeds from the plant; and
d) combining the heterologous protein with a cell culture media.

Another embodiment of the present invention is a method of producing monocot seeds, preferably rice grains, that can express the desired cell culture media component, comprising:

a) transforming a monocot plant cell with a chimeric gene comprising:
(i) a promoter from the gene of a monocot seed storage protein;
(ii) a first DNA sequence, operably linked to the promoter, encoding a monocot seed storage protein; and
(iii) a second DNA sequence, operably linked to the promoter, encoding a cell culture media component,
   wherein the first and second DNA sequences are linked in translation frame and together encode a fusion protein comprising the storage protein and the cell culture media component;
      b) growing a monocot plant from the transformed monocot plant cell for a time sufficient to produce seeds containing the cell culture media component; and
      c) harvesting the seeds from the monocot plant.

Another aspect of the present invention is a method of utilizing monocot seed storage proteins as fusion protein carriers, comprising the above method.

The invention also includes a chimeric gene, comprising:
(i) a promoter from the gene of a monocot seed storage protein;
(ii) a first DNA sequence, operably linked to the promoter, encoding a monocot seed storage protein; and
(iii) a second DNA sequence, operably linked to the promoter, encoding a cell culture media component;
   wherein the first and second DNA sequences are linked in translation frame and together encode a fusion protein comprising the storage protein and the cell culture media component.

The monocot seed storage protein may be at the N-terminal or C-terminal side of the cell culture media component in the fusion protein. It is preferred that the monocot seed storage protein by located at the N-terminal side of the cell culture media component.

Preferably, the monocot plant comprises rice, barley, wheat, rye, corn, millet, triticale, or sorghum. More preferably, the monocot plant is rice.

All publications, patents and patent applications are herein expressly incorporated by reference in their entirety to the same extent as if each individual publication, patent or patent application was specifically and indicated individually to be incorporated by reference in its entirety.

The following examples illustrate but are not intended in any way to limit the scope of the invention.

### EXAMPLES

### Example 1

### Expression of Recombinant Human Growth Factors

In general, expression vectors were constructed using standard molecular biological techniques. The vectors contain a heterologous protein coding sequence for certain growth factors under the control of a rice tissue-specific promoter, as further described below.

The nucleotide sequence of the promoter and the nucleotide sequence of the signal peptide of the rice glutelin-1 (Gt1) gene were cloned based on the published Gt1 gene sequence. The nucleotide sequence of the promoter and the nucleotide sequence of the signal peptide of the rice globulin (Glb) gene were cloned based on the published Glb gene sequence.

A. Generation of Human EGF Expression Vector

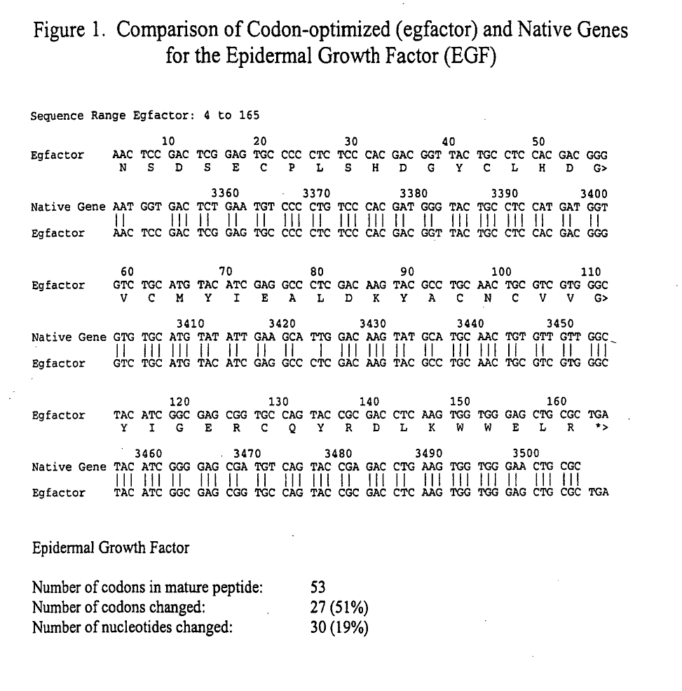

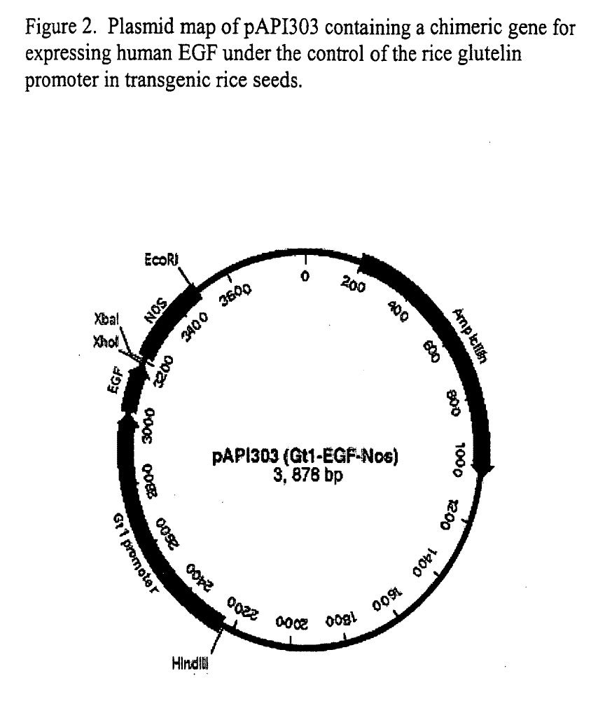

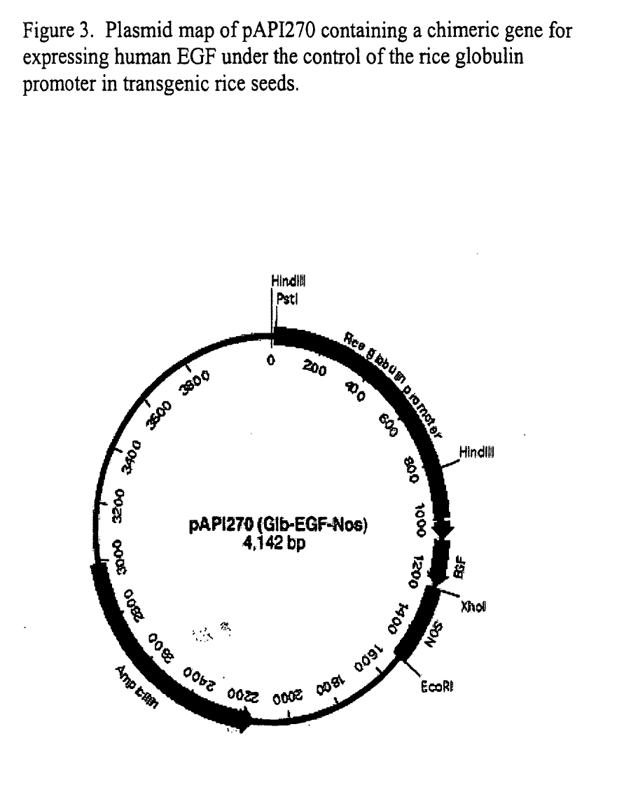

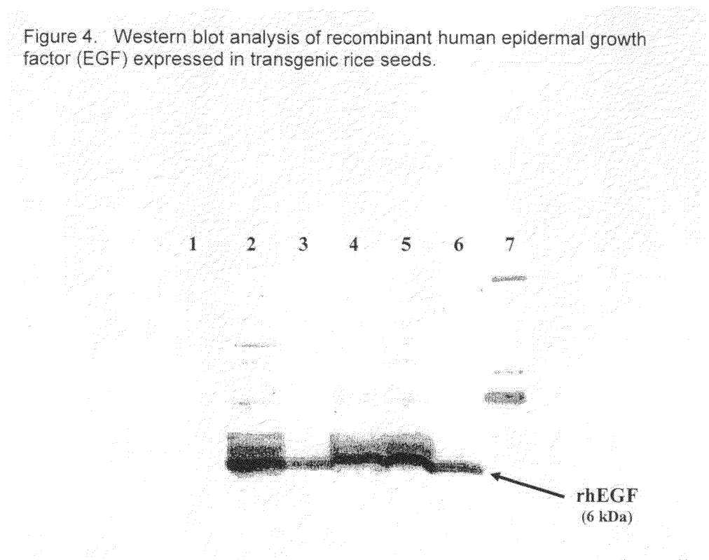

The human EGF gene was codon optimized as shown in FIG. 1, and synthesized by Operon Technologies (CA, USA) (SEQ ID NO: 1). For expression of EGF in rice seeds, the codon optimized gene was operably linked to the rice endosperm specific glutelin (Gt1) promoter, Gt1 signal peptide and NOS terminator in pAPI303 (FIG. 2), and to the rice endosperm specific globulin (Glb) promoter, Glb signal peptide and NOS terminator in pAPI270 (FIG. 3). The transgenic plant expressing EGF was generated, and plant-generated recombinant EGF was detected, as shown in FIG. 4 and as exemplified herein.

B. Generation of Human IGF-I Expression Vector

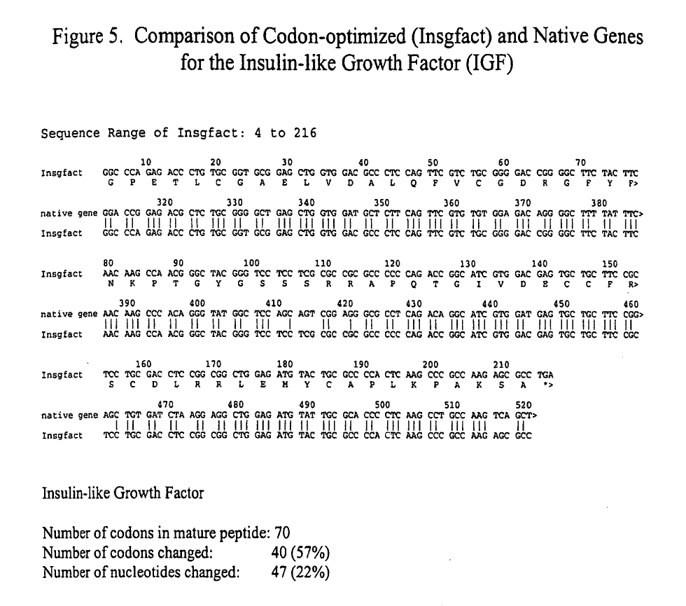

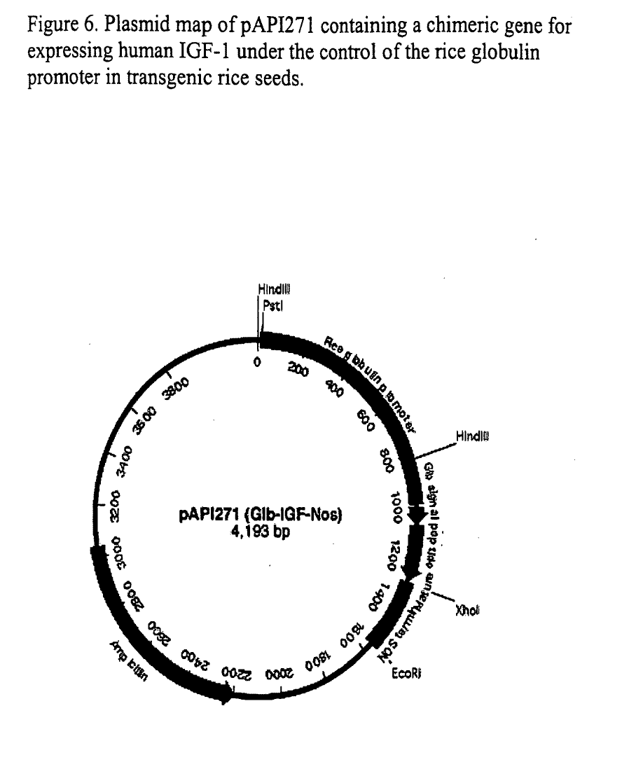

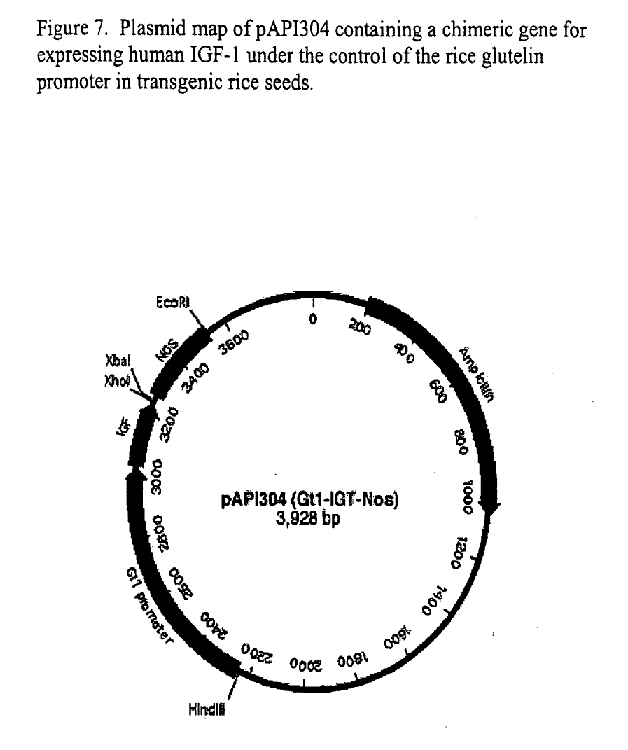

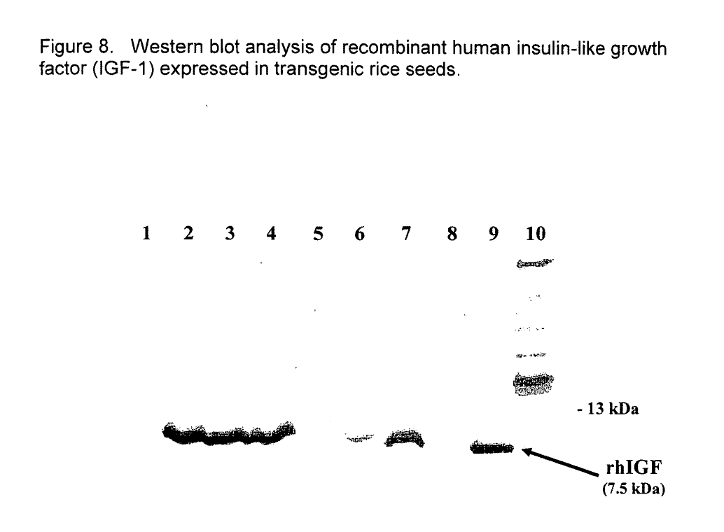

The IGF-I gene was codon optimized as shown in FIG. 5, and synthesized by Operon Technologies (CA, USA) (SEQ ID NO: 3). For expression of IGF-I in rice seeds, the codon optimized gene was operably linked to the rice endosperm specific glutelin (Gt1) promoter, Gt1 signal peptide and NOS terminator in pAPI304 (FIG. 7), and to the rice endosperm specific globulin (Glb) promoter, Glb signal peptide and NOS terminator in pAPI271 (FIG. 6). The transgenic plant expressing IGF-I was regenerated, and plant-generated recombinant IGF-I was detected as shown in FIG. 8 and as exemplified herein.

C. Generation of Human ITF

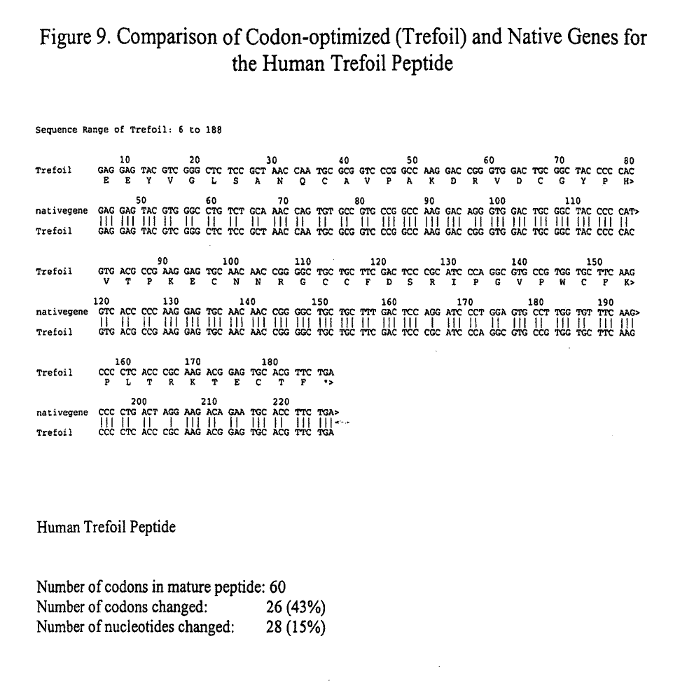

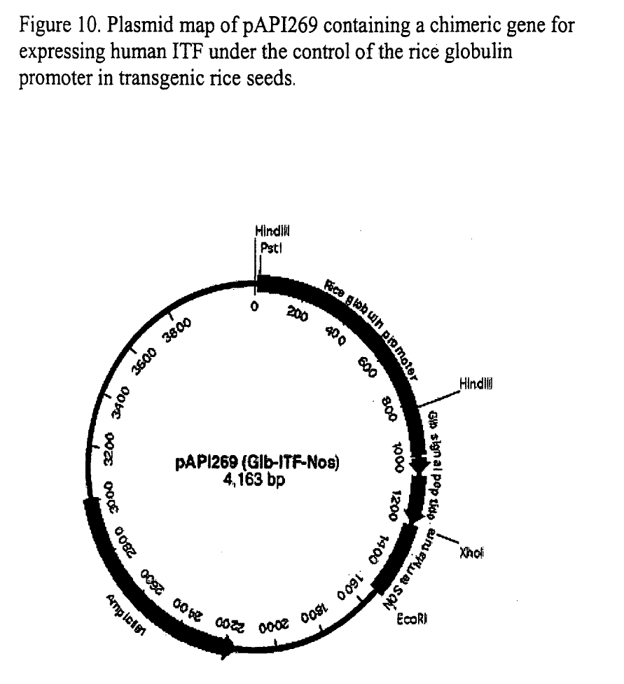

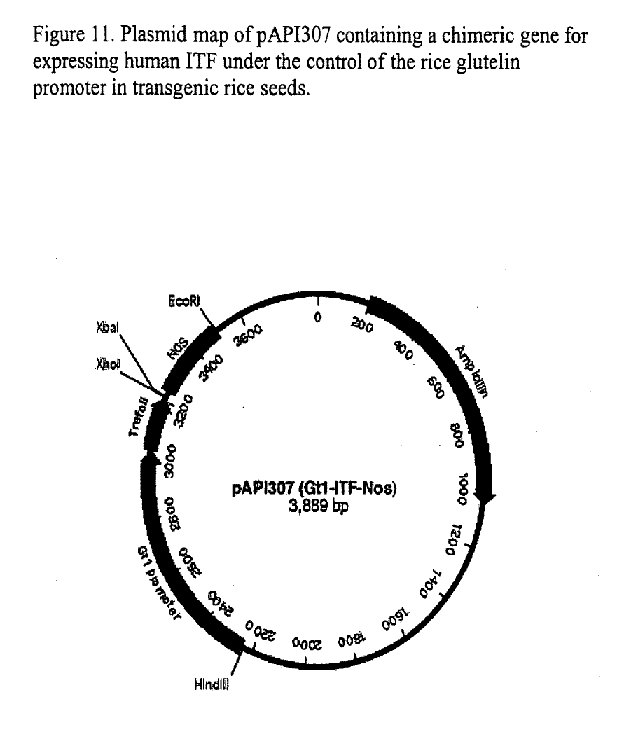

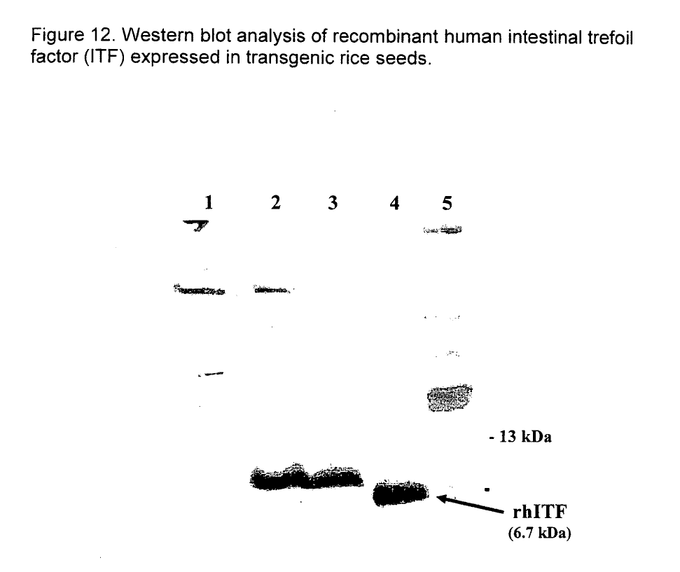

The ITF gene was codon optimized as shown in FIG. 9, and synthesized by Operon Technologies (CA, USA) (SEQ ID NO: 5). For expression of ITF in rice seeds, the codon optimized gene was operably linked to the rice endosperm specific glutelin (Gt1) promoter, Gt1 signal peptide and NOS terminator in pAPI307 (FIG. 11), and to the rice endosperm specific globulin (Glb) promoter, Glb signal peptide and NOS terminator in pAPI269 (FIG. 10). The transgenic plant expressing ITF was generated, and plant-generated recombinant ITF was detected as shown in FIG. 12 and as exemplified herein.

D. Western Blot Analysis for all Growth Factors

Both untransformed (rice variety Taipei 309) and transgenic rice seeds (˜10 pooled R1 seed from individual transgenic plants expressing either EGF, IGF-I or ITF) were ground in 1 ml of 0.35 M NaCl in phosphate buffered saline (PBS), pH 7.4, using an ice-cold mortar and pestle. The resulting extract was centrifuged at 14,000 rpm at 4° C. for 10 min. Cleared supernatant was collected and ˜20 ug of this soluble protein extract was re-suspended in sample loading buffer, and loaded onto a pre-cast 10-20% polyacrylamide tricine gel (Novex) and subjected to SDS-PAGE. After electrophoresis, the gel was blotted to a 0.45 μm nitrocellulose membrane. The membrane was blocked with 5% non-fat dry milk in PBS pH 7.4 for 2 hrs followed by three washes with PBS for 10 min each. A primary rabbit polyclonal antibody prepared against EGF (Sigma), IGF-I (Sigma) or ITF (GI Company) was used at 1:2000 dilution in PBS. Bands were developed using goat anti-rabbit antibody coupled to the BCIP/NBT substrate system (Sigma). Results are shown in FIGS. 4, 8 and 12, respectively.

### Example 2

### Expression of Recombinant Human Lactoferrin

A. An Expression Vector for Human Lactoferrin Expression in Transgenic Rice

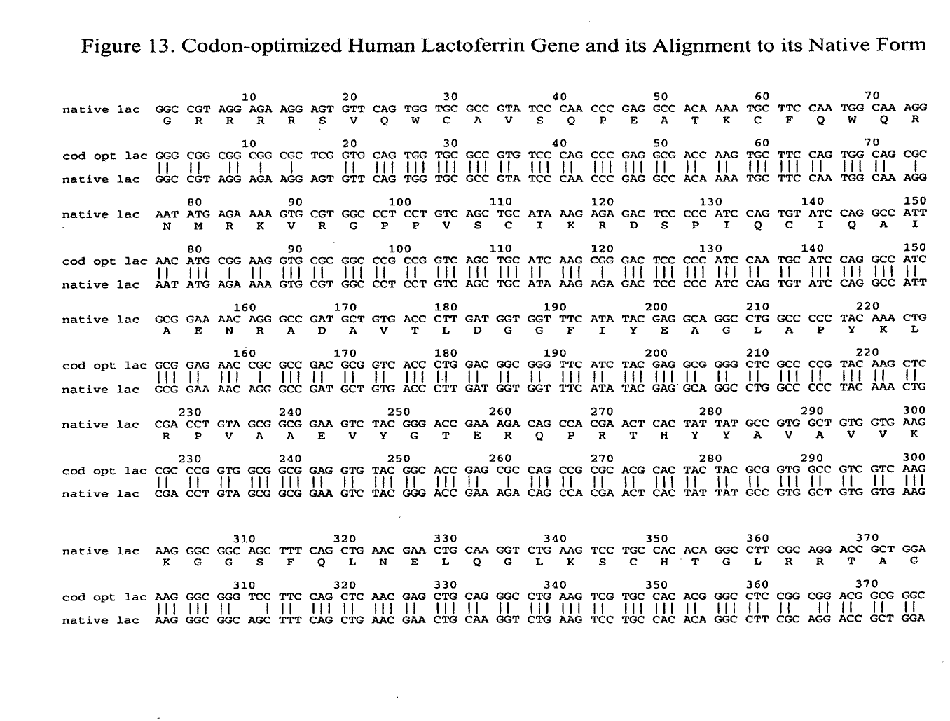

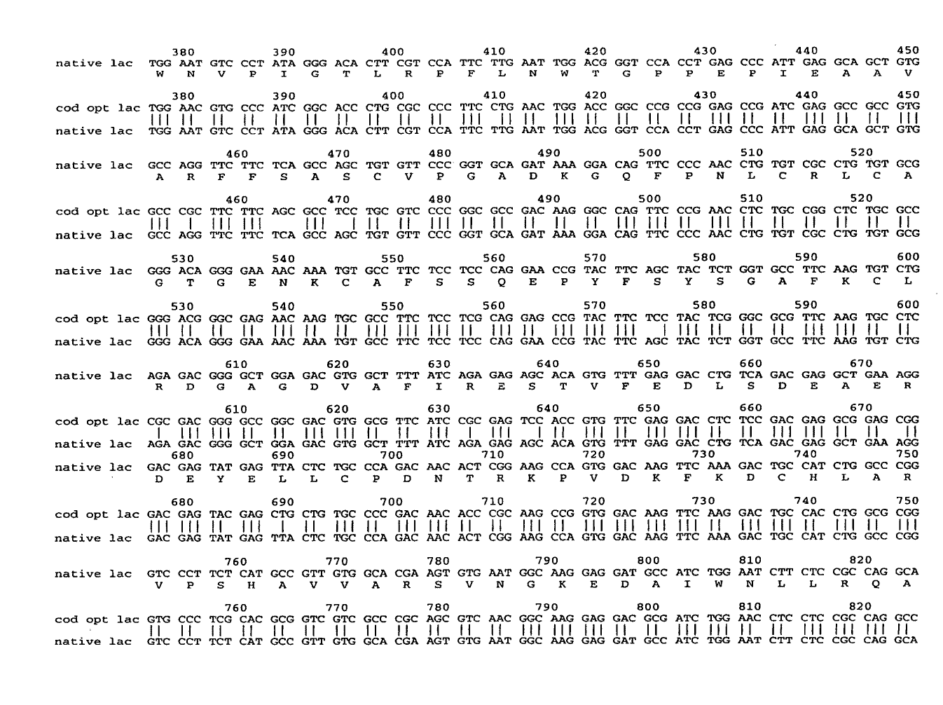

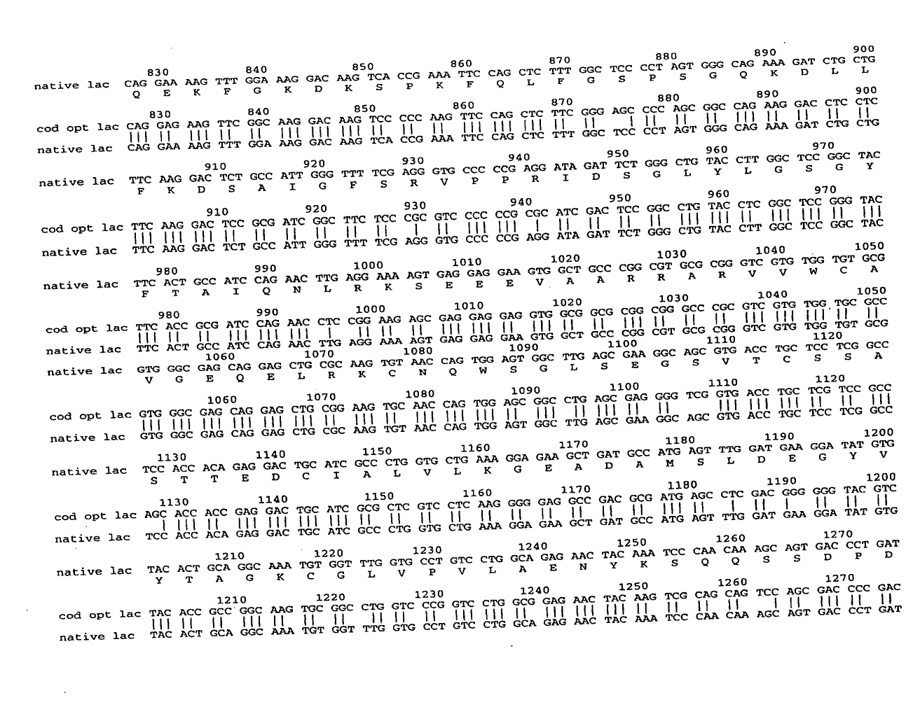

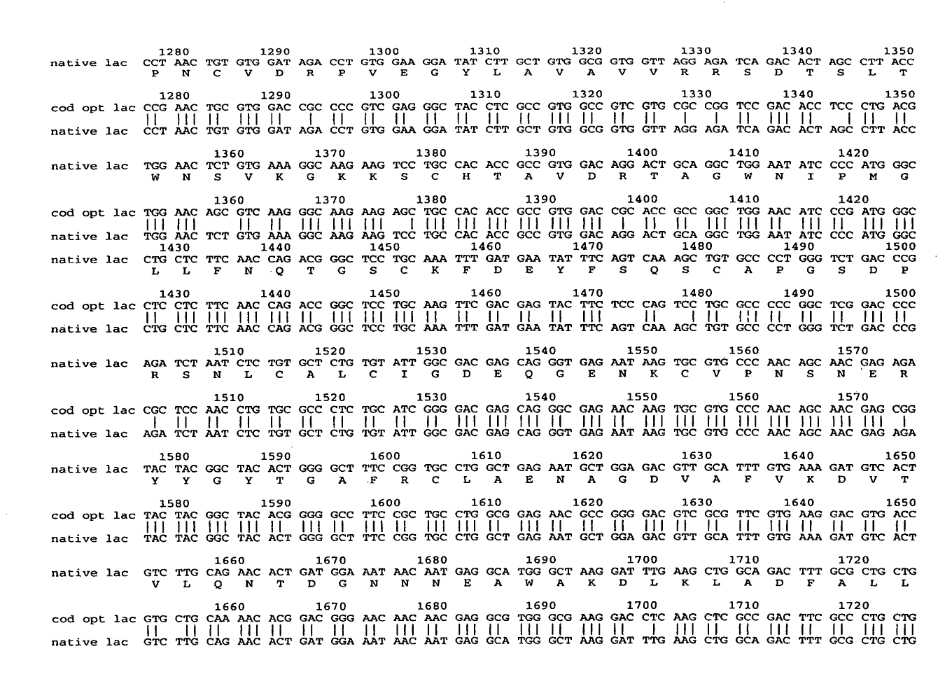

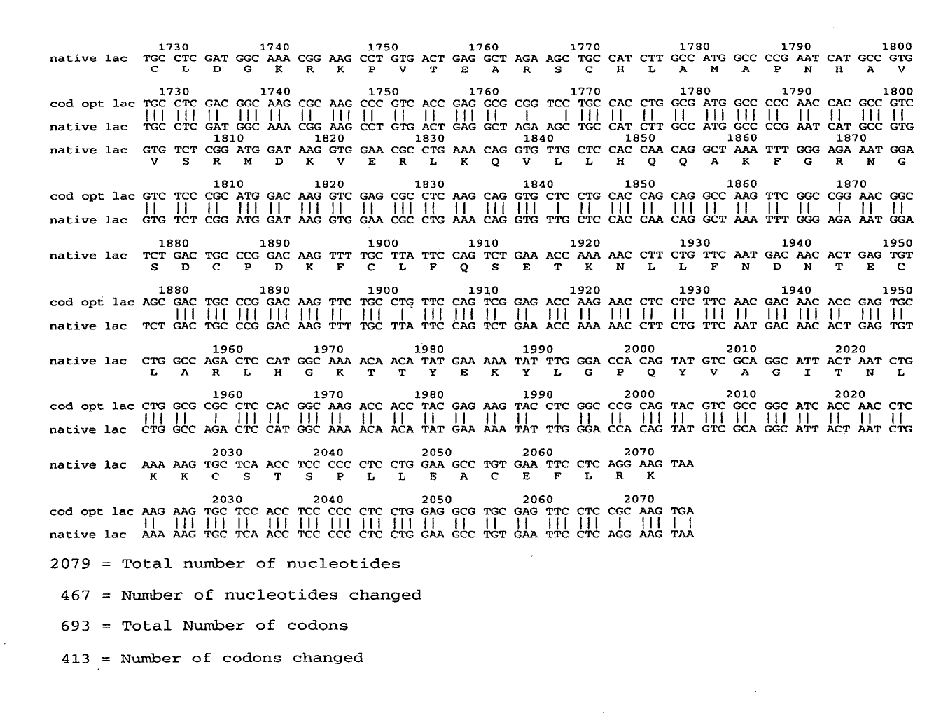

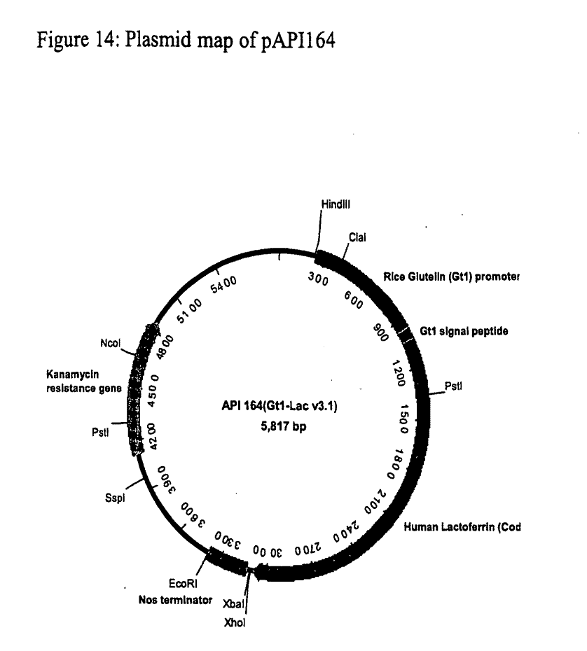

Complete nucleotide sequence of human mammary gland lactoferrin was codon optimized and synthesized by Operon Technologies (CA, USA). Human milk lactoferrin gene (Genbank accession number: HSU07642) was re-synthesized with codons most frequently used in translation of rice seed proteins in order to obtain optimal level of expression (FIG. 13). Although numbers of codons changed accounted for 22.46% of the entire sequence, amino acid composition remains identical to native human lactoferrin. The plasmid containing the codon-optimized gene was called Lac-ger. Lac-ger was digested with SmaI/XhoI and the fragment containing the lactoferrin gene was cloned into pAPI141 that was partially digested with NaeI and completely digested with XhoI. For expression of hLF in rice seeds, the codon-optimized gene was operably linked to the rice endosperm-specific glutelin (Gt1) promoter and NOS terminator. The resulting plasmid was designated pAPI164 (FIG. 14).

B. Production System

Rice variety Taipei 309 (Oryza sativa, Japonica) was selected as the production system for recombinant human lactoferrin (rhLF) and transgenic rice events were generated by the particle bombardment of embryogenic rice calli with the plasmid pAPI164 and a companion marker plasmid containing the hygromycin phosphotransferase gene as a selectable marker. Fully developed, fertile rice plants were obtained by this procedure.

C. High Level Protein Expression of Recombinant Human Lactoferrin in Rice Grain

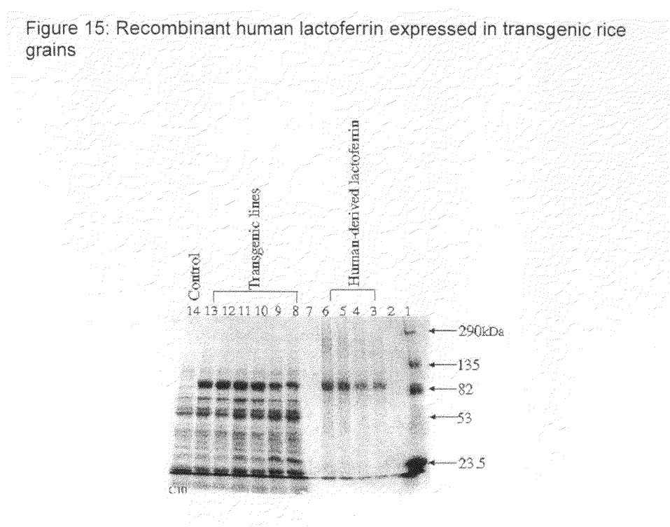

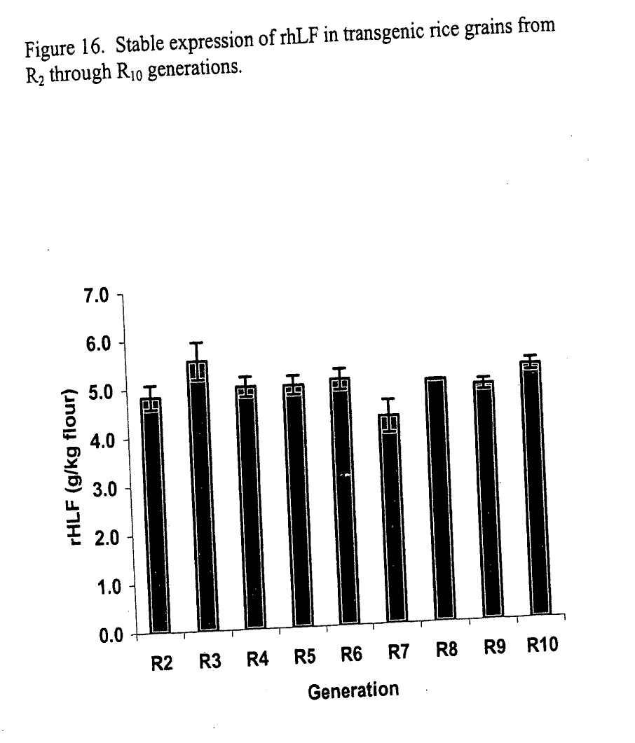

Expression of recombinant human lactoferrin was under the control of the seed maturation-specific promoter Gt1. The high level expression of recombinant human lactoferrin is evident in FIG. 15 as independent transgenic rice events were screened. Total soluble proteins from mature rice seed extracts were run on Laemli gels and stained with Coomassie blue to visualize the proteins. An ˜80 kD recombinant lactoferrin protein was obtained in all transgenic lines as indicated by the stained gel. Expression levels of recombinant human lactoferrin corresponded to 0.5% of seed weight. The stable expression of recombinant human lactoferrin was monitored for 10 generations. The expression level is maintained at approximately 0.5% of seed weight of brown rice (FIG. 16).

### Example 3

### Expression of Recombinant Human Serum Albumin in Rice Grain

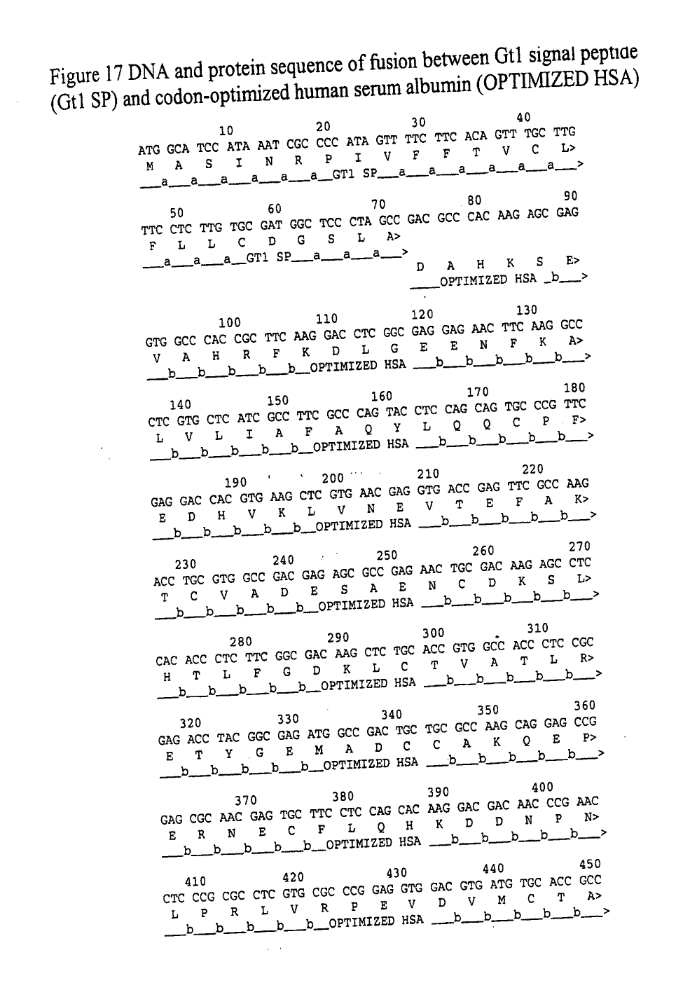

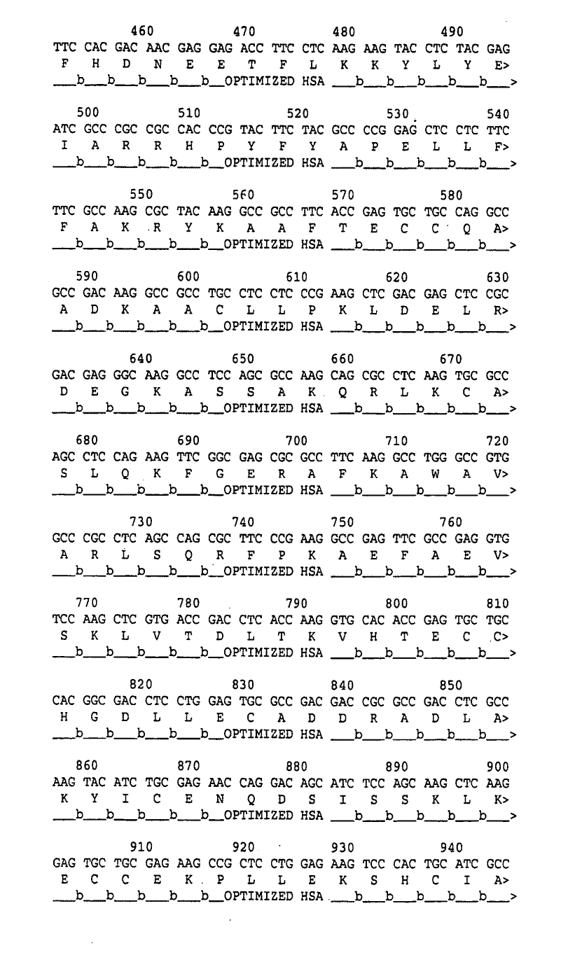

Protein sequences of human serum albumin (HSA) from various data bases were compared. The consensus sequence represented by accession number P02768 (Swiss-Prot) was used as a base for gene codon-optimization for suitable expression of human serum albumin in rice grain (FIG. 17). Gene synthesis was carried out by Blue Heron (Seattle, Wash.) and the synthetic fragment was inserted into pUC based vector to create pUC-HSA. After confirmation of correct DNA and protein sequences, pUC-HSA was digested with MlyI and XhoI. The fragment containing codon-optimized HSA gene was inserted into pAPI405, which had been precut with NaeI and XhoI. Plasmid API405 was a derivative of pAPI141 which included Gt1 promoter, Gt1 signal sequence and a nos terminator.

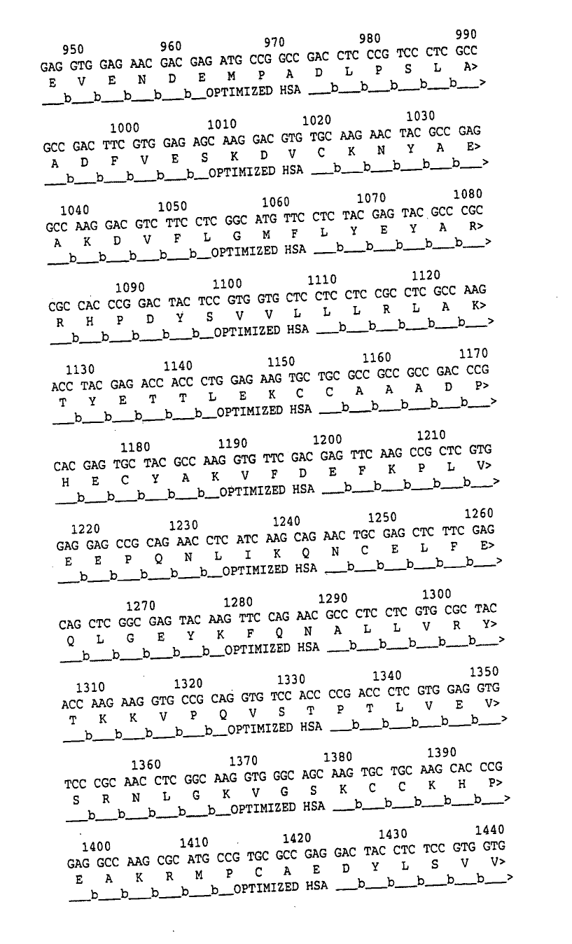

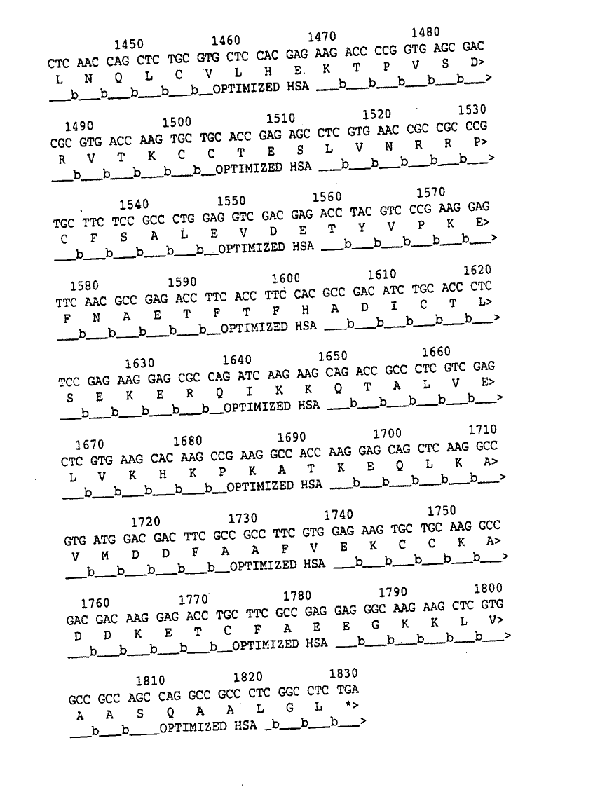

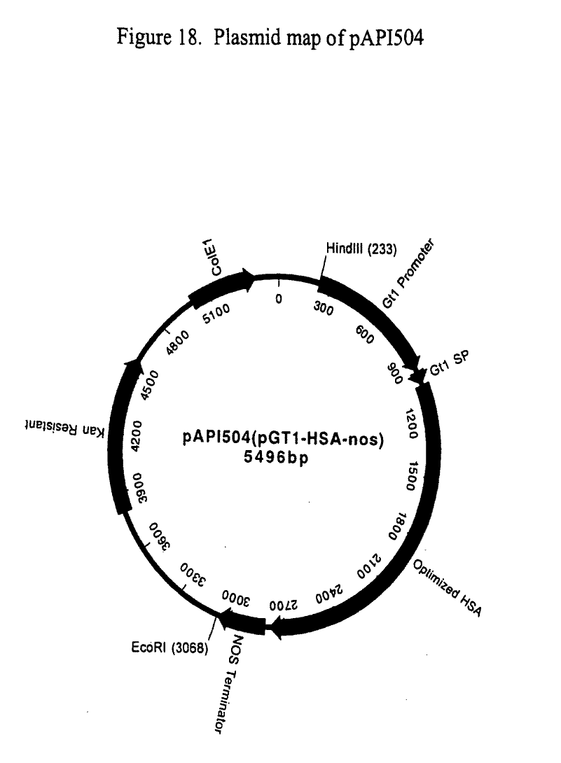

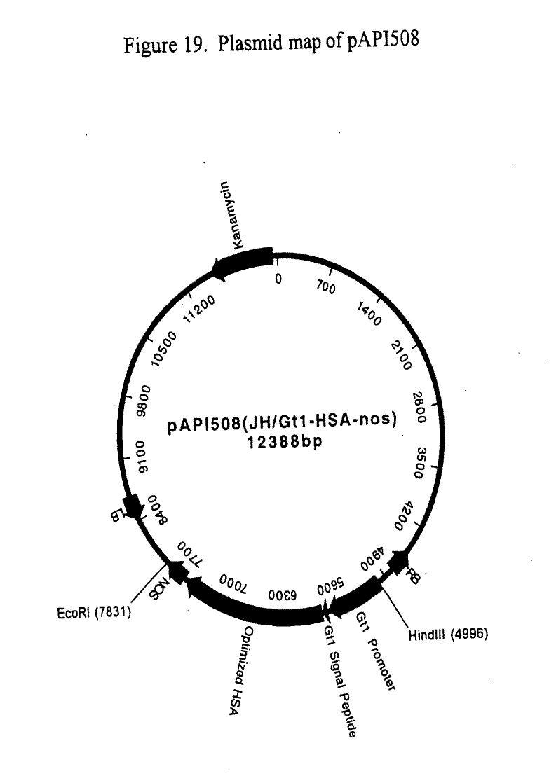

Insertion of MlyI/XhoI fragment into pAPI405 resulted in pAPI504 (FIG. 18). Plasmid API504 was then cleaved with HindIII and EcoRI. The HindIII/EcoRI fragment containing the entire expression cassette, Gt1 promoter, Gt1 signal sequence, codon-optimized HSA gene and nos terminator, was cloned into pJH2600 predigested with the same enzyme resulting in pAPI508 (FIG. 19). Plasmid JH2600 was a shuttle vector between E coli and Agrobacterium. After DNA sequence verification, pAPI508 was moved into Agrobacterium AGL-1 for rice transformation. Plasmid API504 was also used via bombardment-based transformation following the procedure described previously. Upon transformation, transgenic plants were generated and were sent to greenhouse where transgenic R0 plants grew to maturity and set R1 seeds.

To monitor the expression of HSA in rice seeds, 10 R1 seeds from each R0 plant were extracted using 10 mL of extraction buffer (50 mM Tris-HCl pH 8.0. 150 mM NaCl). The supernatant was collected and the expression level from rice extracts was monitored by an ELISA (Bethyl Laboratories, Montgomery, Tex.). The results showed that the HSA expression level in rice transgenic seeds ranges from 0.01 to 0.85% of brown flour weight (0.1 to 8.5 grams/kg flour). The results of the eight events with highest expression levels are shown in the following table (Table 1).

TABLE 1
Recombinant HSA expression level in top eight events

| | Express Level |
|---|---|
| Line Number | (g/kg flour) |
| 508-3 | 5.5 |
| 508-17 | 8.0 |
| 508-71 | 8.7 |
| 508-73 | 4.0 |
| 508-77 | 8.5 |
| 508-83 | 8.5 |
| 508-101 | 3.0 |
| 508-113 | 4.0 |

### Example 4

### Purification of Recombinant Human Lactoferrin as Cell Culture Media Component

To prepare cell culture media supplemented with recombinant human lactoferrin, recombinant human lactoferrin was purified from rice flour. Transgenic rice line (164-12) expressing high levels of rhLF was selected. This line, now named as LF164, was planted two generations per year, alternating field planting in summer and greenhouse planting in winter. For protein purification, paddy rice expressing rhLF was de-hulled (Rice Mill, PS-160, Rimac, Fla.), and then ground to flour (average particle size of 100 mesh) using a hammer mill (8WA, Schutte-Buffalo, N.Y.).

Protein extraction from transgenic flour was performed by mixing two kg of rice flour and 20 L of extraction buffer (0.02 M sodium phosphate pH 6.5 and 0.3 M sodium chloride) in a 50 L tank for 1 h. At the end of the mixing period, the suspension was allowed to settle overnight or centrifuged at 3750 rpm. In both cases, the supernatant was filtered through a plate and frame filter press (Ertel Alsop, 8S, NY) using M-05 and M-70 cellulose/perlite-based filters (Ertel Alsop, NY), respectively.

The filtrate containing rhLF and other rice flour soluble proteins was loaded onto an ion exchange column for further purification. An INDEX 200/500 process column (Amersham Pharmacia Biotech, NJ) packed with SP-Sepharose fast flow (Amersham Pharmacia Biotech, NJ) was used. The column was run at a linear flow rate of 150-200 cm/h. Packing, cleaning and testing of the resin bed was executed per manufacturer's instruction (HETP Test). The filtrate was loaded on to the resin at a linear velocity of 175 cm/h and washed with 0.02 M sodium phosphate buffer (pH 6.5) containing 0.3 M NaCl until the A₂₈₀ returned to baseline. Recombinant hLF was eluted using 20 mM sodium phosphate buffer (pH 6.5) containing 0.8 M NaCl. The washing and elution were performed at 200 cm/h and 150 cm/h, respectively.

A Centramate module (Pall Biopharmaceutical, MA) with 1 ft² 50 kDa polyethersulfone (Pall Biopharmaceutical, MA) membrane was used for concentration and desalting (ultrafiltration) of eluted hLF. The filtration was performed at a cross flow rate of approximately 1.5 L/min and an average transmembrane pressure of 10 psig. The eluted rhLF was concentrated and desalted to a final volume of 0.25 L and then lyophilized dry. Usually, about 3 grams of purified recombinant human lactoferrin was recovered from one kilogram of transgenic rice flour.

The recombinant human lactoferrin purified from rice flour is approximately 50% saturated with iron (partial-lactoferrin). The 50% saturated recombinant human lactoferrin was then made >90% iron saturated by iron up taking treatment, resulting in holo-lactoferrin and was made <10% iron saturated by acid treatment to remove bound iron resulting in apo-lactoferrin. The purified lactoferrins (holo-, partial- and apo-) were used as cell culture media components.

### Example 5

### Cell Culture Media Supplemented with Recombinant Human Lactoferrin Promotes Cell Growth

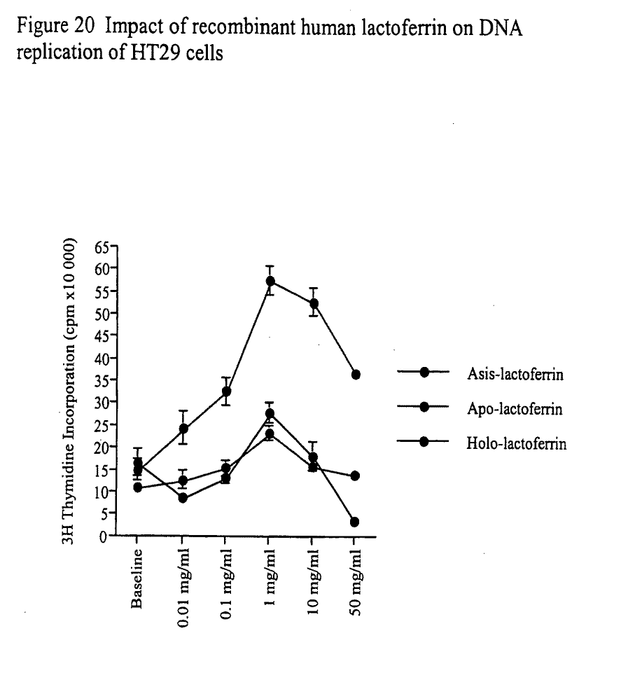

Recombinant human lactoferrin was added to minimal essential media (MEM) at various concentrations. This allows the test of growth factor effect of lactoferrin. The human colonic cancer cell line, HT-29, were maintained in MEM basic media containing 10% fetal calf serum (FCS). Lactoferrin samples were subsequently tested under serum-free conditions (MEM without supplementation of fetal calf serum). To assess the percentage of cells entering DNA synthesis, [³H]-thymidine (2 μCi/well) was included twenty four hours after the addition of lactoferrin samples and cells were left for a further 24 h. For each condition, the stimulatory or inhibitory effect of the lactoferrin solutions was measured in duplicate in six separate wells. Cell viability, determined by the ability to exclude 0.2% trypan blue, was greater than 90%. Proliferation assays reveal a typical ‘bell shaped’ dose response curve (FIG. 20). Results demonstrated these responses to lactoferrin addition and show that maximal activity of lactoferrin was at a final concentration of 1 mg/ml, for all three forms of lactoferrin. Furthermore, of the three forms, the holo-lactoferrin showed the greatest proliferative activity (FIG. 20).

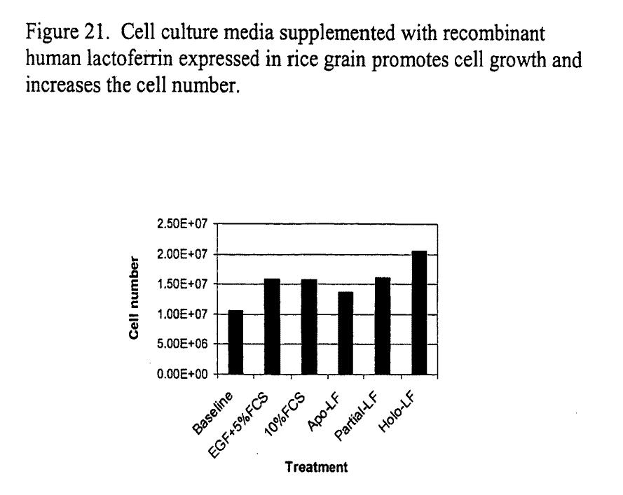

To determine cell viability, HT-29 Cells were seeded in 24-well plates at 5×10⁵ cells per well in MEM supplemented with 5% fetal calf serum (FCS); this resulted in an attached cell number of >95% per well 24 h later (as assessed by trypsinization and direct counting using a Neubauer haemocytometer). At this time, the medium was changed in identically seeded wells to one containing MEM with 5% FCS alone or MEM with 5% FCS plus EGF, or additional 5% FCS or various forms of Lactoferrin (i.e., apo-, partial- or holo-) at a concentration of 1 mg/ml for a further 48 h. At the end of this period, cell numbers were assessed. To ensure that any cells present in the supernatant at this time were included in the total cell count, supernatants were collected, centrifuged at 13,000 rpm (12,000 g) for 5 min and added to the cells following trypsinization. Result shows that recombinant human lactoferrin derived from rice promote cell growth. There were twice as many cells in wells supplemented with holo-lactoferrin as that in wells without lactoferrin (FIG. 21). Both thymidine incorporation and cell count experiments indicate that recombinant human lactoferrin from rice can be added as a component to cell culture media for fast cell growth (FIGS. 20 and 21).

### Example 6

### Cell Culture Media Supplemented with Recombinant Human Lactoferrin Promotes Hybridoma Cell Growth

Recombinant human holo-lactoferrin was added to serum free media at various concentrations ranging from 5 to 250 mg/ml for hybridoma cell culture. Since the recombinant human lactoferrin is iron-saturated, holo-lactoferrin can provide iron for cell growth. The hybridoma cell line AE1 (ATCC) was maintained in DMEM basic media containing 5% fetal bovine serum (FBS). Lactoferrin was tested under serum-free conditions (AFM6, KC Bio, Kansas) without supplementation of fetal bovine serum. The cells were subcultured from 5% FBS to serum free media over multiple passages. At each subculture, the cells were analyzed for total cell count and viability. In order to demonstrate that recombinant human lactoferrin can replace transferrin derived from animal source, transferrin of equal concentration was added to AFM6 media as the control. The results demonstrated that the recombinant human holo-lactoferrin can be used to replace animal-derived transferrin in cell culture media for hybridoma cell growth.

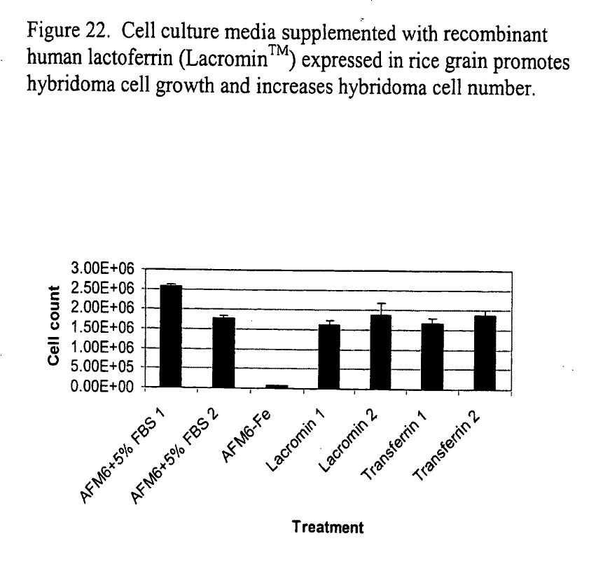

To determine the ability of holo-lactoferrin to promote cell growth, AE1 cells were sub-cultured from media containing 5% FBS to serum free media in T25 stationary flasks. The serum free media was supplemented with either animal derived transferrin; which is commonly used in the industry, or recombinant human holo-lactoferrin (LACROMIN™). This allows for a direct comparison between recombinant human holo-lactoferrin and animal derived transferrin. Each protein test was performed in triplicate with seeding densities of 5×10⁵ cells per ml. To the subculture, the cells were collected by centrifugation and then seeded into fresh media with identical concentrations of either transferrin or recombinant human holo-lactoferrin (LACROMIN™). Each flask was analyzed on day 1 and day 3, following the subculture, for cell number and viability. An average cell count is given in FIG. 22 for two passages. As can be seen, the cell viability and cell number when using recombinant human holo-lactoferrin are much higher than that without lactoferrin and equivalent to that of animal derived transferrin, indicating that plant-derived recombinant human holo-lactoferrin can promote hybridoma cell growth and replace animal derived transferrin.

The same analysis was carried out with another hybridoma cell line, L243. As can be seen in Table 2. LACROMIN™ (rhLF) can promote cell growth as well as human transferrin (TF). L243 is also an IgG producing cell line. Samples were taken for IgG production. In medium containing LACROMIN™, more IgG is produced than in medium containing transferrin.

TABLE 2
Effect of human transferrin (TF) and recombinant
human lactoferrin (rhLF) on hybridoma growth
and monoclonal antibody production.

| | TF/rhLF | Cell count (×10⁶) | | IgG level (ug/ml) | |
|---|---|---|---|---|---|
| Treatment | (ug/ml) | Mean | SD | Mean | SD |
| Control | 0 | 0.39 | 0.18 | 15.78 | 5.63 |
| TF | 5 | 0.70 | 0.25 | 34.10 | 8.44 |
| rhLF | 5 | 0.63 | 0.10 | 43.58 | 8.24 |
| TF | 25 | 0.78 | 0.24 | 31.93 | 7.10 |
| rhLF | 25 | 0.81 | 0.20 | 36.10 | 7.54 |
| TF | 100 | 0.86 | 0.33 | 28.58 | 5.82 |
| rhLF | 100 | 0.78 | 0.26 | 29.73 | 4.28 |
| 5% FBS | 0 | 0.98 | 0.12 | 23.88 | 5.50 |

### Example 7

### Purification and Characterization of Recombinant Human Serum Albumin

To purify rHSA from rice grain, rice grain expressing rHSA was dehusked, ground to flour and mixed for 30 min. with rHSA extraction buffer (Sodium Acetate, pH 4.9). After clarification by filtration, the filtrate was loaded onto an anion-exchange column (Q-Sepharose; GE Healthcare) at a rate of approximately 150 cm/hr. Once loaded, the resin was then washed with equilibration buffer to remove any unbound protein. The eluted rHSA is approximately 90% pure as analyzed by SDS-PAGE. Blue-Sepharose (GE Healthcare) can then be used to increase the purity from 90% to approximately 98% based on scanning analysis of SDS-PAGE gels. The rHSA is then concentrated and desalted via ultrafiltration prior to freeze drying.

Purified rHSA was subjected to a range of biochemical and biophysical characterizations for comparison to pHSA (Plasma-derived HSA) (Table 3). These tests indicate that rHSA expressed in rice is equivalent to pHSA.

TABLE 3
Properties of rHSA as compared to pHSA

| Property | pHSA | rHSA |
|---|---|---|
| N-terminal sequence | DAHKSE | DAHKSE |
| Glycosylation | None | None |
| SDS-PAGE/Western | ~66 kDa | ~66 kDa |
| Molecular mass (MALDI) | 66.9-68.1 kDa | 66.8-67.9 kDa |
| Isoelectric focusing point | pI 5.3 | pI 5.3 |
| Ligand binding | Yes | Yes |
| Thermal stability | Midpoint 65° C. | Midpoint 65° C. |
| Esterase activity | Yes | Yes |
| Protease sensitivity | Same as each other | Same as each other |

### Example 8

### Cell Culture Media Supplemented with Recombinant Human Serum Albumin Promotes Hybridoma Cell Growth in Reduced-Serum Media

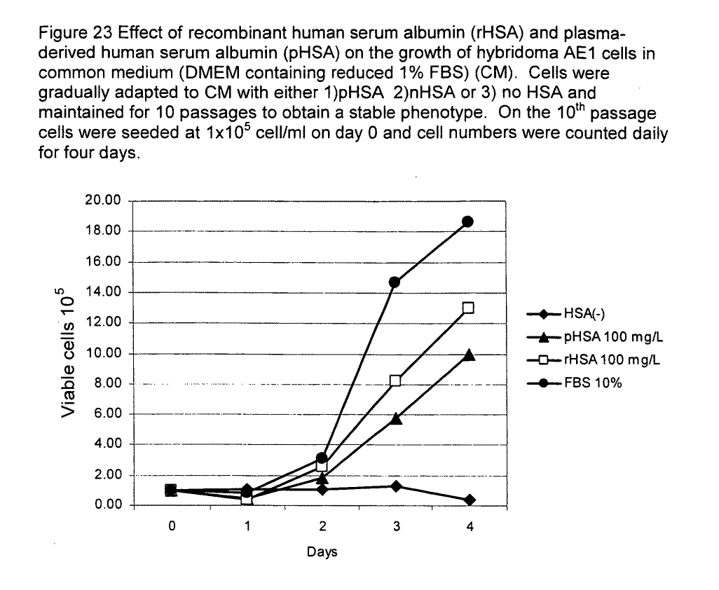

The effectiveness of recombinant human serum albumin from rice (rHSA) to stimulate cell growth similar to plasma derived human serum albumin (pHSA) was tested. Basal medium DMEM supplemented with 10 μg/ml insulin, 5.5 μg/ml transferrin, 6.7 ng/ml sodium selenite, 0.20 mg/ml ethanolamine and 2 mM Glutamax (Gibco) was used as common medium (CM). Hybridoma AE1 stocks in CM+10% fetal bovine serum (FBS), were divided into four lineages: 1) CM+10% FBS as positive control 2) CM without HSA as negative control 3) CM+100 mg/L rHSA as experimental, and 4) CM+100 mg/L plasma-derived HSA (pHSA) as comparative control. Each lineage was then gradually adapted to 1% FBS (reduced serum) by sequential passage of 1×10⁵ cell/ml into medium containing 50% reduced FBS. At 1% FBS, each lineage was subsequently passaged 10 times to allow for full adaptation to growth under reduced-serum (1% FBS) conditions. FIG. 23 shows cell growth curves of the 4 lineages during the 10th passage under CM+1% FBS. It was found that AE1 hybridoma cells grew much faster and reached a higher density in CM rHSA than in CM without any HSA (FIG. 23). It was also found that cells grown in media with rHSA supplementation grew equivalently or better than cells grown in media with plasma derived HSA. This result indicates that rHSA from rice is functionally equivalent to pHSA in enhancing hybridoma cell growth in reduced-serum media.

### Example 9

### Cell Culture Media Supplemented with Recombinant Human Serum Albumin Promotes Hybridoma Cell Growth in Serum-Free Media

In order to demonstrate that plant derived recombinant human serum albumin functions equivalently to albumin derived from animals to support cell growth, the growth of AE1 hybridoma cells in supplemented serum-free media (SFM) was tested. SFM was prepared that was supplemented with either recombinant human serum albumin or plasma derived human serum albumin at similar concentrations (100 mg/L). SFM without supplemental albumin was prepared as a negative control. This approach allows for direct comparison between plant derived recombinant human serum albumin (CELLASTIM™) and plasma derived human serum albumin in promoting cell growth. SFM consisted of DMEM/F12 basal media supplemented with 10 μg/ml insulin, 5.5 μg/ml transferrin, 6.7 ng/ml sodium selenite, 0.20 mg/ml ethanolamine and 2 mM Glutamax (Gibco). To reach serum-free growth conditions, AE1 Hybridoma cells (ATCC # HB-72) were first adapted to SFM without FBS by a serial adaptation procedure. Briefly, AE1 grown in SFM/10% FBS supplemented with recombinant human serum albumin or plasma derived human serum albumin or no HSA, were successively sub-cultured at 1×10⁵ cells/ml into SFM with 50% reduced serum from the previous culture. The SFM/0.25% FBS cultures were subcultured directly into SFM without FBS. Six successive subcultures followed in SFM to allow cells to fully adapt to the respective SFM without FBS.

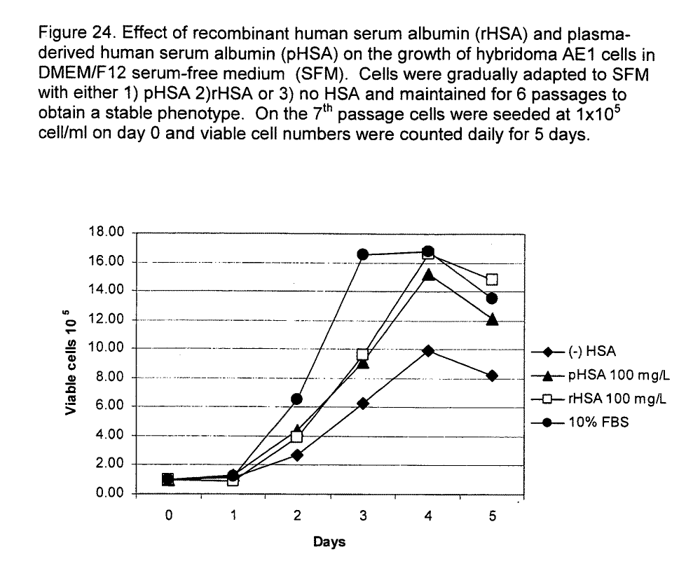

AE1 hybridoma cells grown for 6 passages in SFM containing 100 mg/L recombinant human serum albumin or 100 mg/L plasma derived human serum albumin or no HSA were sub-cultured 6-well plates at 1×10⁵ cells/ml. The number of viable cells and percent viability was determined daily to examine growth kinetics of the cultures. The subcultures were performed in duplicate to ensure an accurate viable cell count and analysis. FIG. 24 shows that AE1 hybridoma cell grew faster and that the maximum number of viable cells was higher in medium supplemented with plant derived recombinant human serum albumin and equivalent to that of plasma derived human serum albumin. Thus, this example shows that plant derived recombinant human serum albumin can promote cell growth in serum-free media and replace animal derived albumin for use in tissue culture.

The invention now being fully described, it will be apparent to one of ordinary skill in the art that many changes and modifications can be made thereto without departing from the spirit or scope of the appended claims.

1. A cell culture media, comprising at least one plant-produced heterologous protein as a cell culture media component.

2. The cell culture media of claim 1, wherein the plant is a monocot plant.

3. The cell culture media of claim 2, wherein the monocot plant is rice, barley, wheat, rye, corn, millet, triticale, or sorghum.

4. The cell culture media of claim 3, wherein the monocot plant is rice.

5. The cell culture media of claim 1, wherein the plant is a dicot plant.

6. The cell culture media of claim 1, wherein the heterologous protein is a plant protein.

7. The cell culture media of claim 1, wherein the heterologous protein is a non-plant protein.

8. The cell culture media of claim 7, wherein the non-plant protein is an animal protein.

9. The cell culture media of claim 7, wherein the non-plant protein is a human protein.

10. The cell culture media of claim 1, wherein the protein is selected from the group consisting of growth factors, lactoferrin, transferrin, serum albumin, insulin, growth hormone, and fractions thereof, fibronectin attachment factor, lamin attachment factor, collagenase, platelet derived growth factor, brain-derived neutrophic factor, glial-derived neurotrophic factor, thymic factors, haptocorin, lactahedrin, lactoperoxidase, alpha-fetoprotein, immunoglobin, or alpha-lactalbumin.

11. The cell culture media of claim 10, wherein the growth factors are epidermal growth factors, keratinocyte growth factors, insulin-like growth factors, intestinal trefoil factors, transforming growth factors, granulocyte colony-stimulating factors, nerve growth factors, fibroblast growth factors, or biologically active fragments thereof.

12. The cell culture media of claim 1, wherein the cell culture media is a reduced serum, serum-free medium, or chemically defined media.

13. The cell culture media of claim 1, wherein the cell culture media is a complete media, basal media, or basal media supplemented with a cell culture media component.

14. A method for producing an enhanced cell culture media, comprising producing at least one heterologous protein in a plant cell and combining the at least one plant-produced heterologous protein with a cell culture media.

15. A cell culture media produced by a process comprising producing at least one heterologous protein in a plant cell and combining the at least one plant-produced heterologous protein with a cell culture media.

16. In a cell culture media having one or more proteinaceous cell culture media components, the improvement comprising at least one plant-produced heterologous protein as a cell culture media component, wherein the heterologous protein is produced by the following process:
a) transforming a plant cell with a chimeric gene comprising:
(i) a promoter from the gene of a seed storage protein;
(ii) a first DNA sequence, operably linked to the promoter, encoding a signal sequence of a seed storage protein, and
(iii) a second DNA sequence, operably linked to the promoter, encoding the heterologous protein, wherein the first and second DNA sequences are linked in translation frame and together encode a fusion protein comprising the storage protein and the heterologous protein; and
b) growing a plant from the transformed plant cell for a time sufficient to produce seeds containing the heterologous protein.

17. The method of claim 17, further comprising purifying the heterologous protein from the harvested seeds.

18. The method of claim 17, wherein the heterologous protein constitutes at least about 3.0% of the total soluble protein in the harvested seeds.

19. A method for producing a cell culture media having at least one plant-produced heterologous protein as a cell culture media component, the method comprising:
a) transforming a plant cell with a chimeric gene comprising:
(i) a promoter from the gene of a seed storage protein;
(ii) a first DNA sequence, operably linked to the promoter, encoding a signal sequence of a seed storage protein, and
(iii) a second DNA sequence, operably linked to the promoter, encoding the heterologous protein, wherein the first and second DNA sequences are linked in translation frame and together encode a fusion protein comprising the storage protein and the heterologous protein;
b) growing a plant from the transformed plant cell for a time sufficient to produce seeds containing the heterologous protein;
c) harvesting the seeds from the plant; and
d) combining the heterologous protein with a cell culture media.
